(19) United States
(12) Patent Application Publication
(10) Pub. No.: US 2011/0059983 A1
(43) Pub. Date: Mar. 10, 2011

(54) USE OF SUBSTITUTED OXINDOLE DERIVATIVES FOR THE TREATMENT AND PROPHYLAXIS OF PAIN

(75) Inventors: Wilfried Braje, Ludwigshafen (DE); Marcel Van Gaalen, Ludwigshafen (DE); Anton Bespalov, Ludwigshafen (DE); Charles David Mills, Highland Park, IL (US)

(73) Assignees: Abbott GmbH & Co. KG, Wiesbaden (DE); Abbott Laboratories, Abbott Park, IL (US)

(21) Appl. No.: 12/797,385

(22) Filed: Jun. 9, 2010

Related U.S. Application Data
(60) Provisional application No. 61/185,778, filed on Jun. 10, 2009.

(51) Int. Cl. A61K 31/496; A61K 31/4545; C07D 401/14; A61P 29/00

(52) U.S. Cl. 514/253.09; 514/316; 544/364; 546/187

(57) ABSTRACT

The present invention relates to the use of substituted oxindole derivatives of formula I as defined in the claims and description for the treatment or prophylaxis of pain.

### CROSS-REFERENCE TO RELATED APPLICATION

This claims priority to U.S. Provisional Patent Application No. 61/185,778, filed Jun. 10, 2009, the contents of which are hereby incorporated by reference.

The present invention relates to the use of substituted oxindole derivatives of formula I as defined below for the treatment or prophylaxis of pain.

Pain can be classified as acute and chronic pain. Acute pain and chronic pain differ in their etiology, pathophysiology, diagnosis and treatment.

Acute pain, which occurs following tissue injury, is self-limiting, serves as an alert to ongoing tissue damage and following tissue repair it will usually subside. There are minimal psychological symptoms associated with acute pain apart from mild anxiety. Acute pain is nociceptive in nature and occurs following chemical, mechanical and thermal stimulation of A-delta and C-polymodal pain receptors.

Chronic pain, on the other hand, serves no protective biological function. Rather than being the symptom of tissue damage it is a disease in its own right. Chronic pain is unrelenting and not self-limiting and can persist for years, perhaps decades after the initial injury. Chronic pain can be refractory to multiple treatment regimes. Psychological symptoms associated with chronic pain include chronic anxiety, fear, depression, sleeplessness and impairment of social interaction. Chronic non-malignant pain is predominantly neuropathic in nature and involves damage to either the peripheral or central nervous systems.

Acute pain and chronic pain are caused by different neuro-physiological processes and therefore tend to respond to different types of treatments. Acute pain can be somatic or visceral in nature. Somatic pain tends to be a well localised, constant pain and is described as sharp, aching, throbbing or gnawing. Visceral pain, on the other hand, tends to be vague in distribution, paroxysmal in nature and is usually described as deep, aching, squeezing or colicky in nature. Examples of acute pain include post-operative pain, pain associated with trauma and the pain of arthritis. Acute pain usually responds to treatment with opioids or non-steroidal anti-inflammatory drugs.

Chronic pain, in contrast to acute pain, is described as burning, electric, tingling and shooting in nature. It can be continuous or paroxysmal in presentation. The hallmarks of chronic pain are chronic allodynia and hyperalgesia. Allodynia is pain resulting from a stimulus that normally does not ellicit a painful response, such as a light touch. Hyperalgesia is an increased sensitivity to normally painful stimuli. Primary hyperalgesia occurs immediately within the area of the injury. Secondary hyperalgesia occurs in the undamaged area surrounding the injury. Examples of chronic pain include complex regional pain syndromes, peripheral neuropathies, mechanical nerve injury and severe pain associated with diseases such as cancer, metabolic disease, neurotropic viral disease, neurotoxicity and multiple sclerosis. Chronic pain tends to be only partially responsive to treatment with opioid drugs.

Although opioids are cheap and effective, serious and potentially life-threatning side effects occur with their use, most notably respiratory depression and muscle rigidity. In addition the doses of opioids which can be administered are limited by nausea, emesis, constipation, pruritis and urinary retention, often resulting in patients electing to receive sub-optimal pain control rather than suffer these distressing side-effects. Furthermore, these side-effects often result in patients requiring extended hospitalisation. Opioids are highly addictive and are scheduled drugs in many territories.

Efforts continue therefore to find new treatments for pain management, in particular treatment of chronic pain. Moreover treatments of pain management are needed which are safe as well as effective. It was therefore an object of the present invention to provide compounds for the treatment or prophylaxis of pain, in particular of chronic pain.

The object is achieved by the use of compounds of the formula I

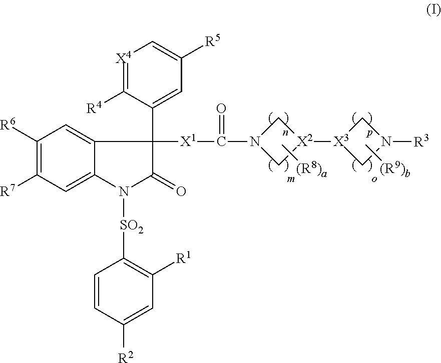

in which
R¹ and R² are independently of one another hydrogen, C₁-C₃-alkyl, C₁-C₃-fluoroalkyl, C₁-C₃-alkoxy, C₁-C₃-fluoroalkoxy, halogen or CN;
R³ is hydrogen or C₁-C₄-alkyl;
R⁴ is methoxy, ethoxy, fluorinated ethoxy or isopropoxy;
R⁵ is hydrogen or methyl;
R⁶ is Br, Cl, For CN;
R⁷ is hydrogen, Cl, F or CN;
R⁸ and R⁹ are independently of one another C₁-C₃-alkyl or C₁-C₃-fluoroalkyl;
X¹ is O, NH or CH₂;
X² and X³ are N or CH, with the proviso that X² and X³ are not simultaneously N;
X⁴ is N or CH;
a and b are independently of one another 0, 1 or 2; and
m, n, o and p are independently of one another 1, 2 or 3;
or of a pharmaceutically acceptable salt thereof or of a prodrug thereof;
   for preparing a medicament for the treatment or prophylaxis of pain.

Preferably, the invention relates to the use of compounds of formula I or of a pharmaceutically acceptable salt thereof or of a prodrug thereof for preparing a medicament for the treatment or prophylaxis of chronic pain and in particular of neuropathic pain.

The invention also relates to a compound of formula I or of a pharmaceutically acceptable salt thereof or of a prodrug thereof for treating or preventing pain.

The invention also refers to a method for treating or preventing pain, preferably chronic pain and in particular neuropathic pain, which comprises administering an effective amount of at least one compound of the formula I as defined above or below or of at least one pharmaceutically acceptable salt or a prodrug thereof or of a pharmaceutical composition containing at least one compound I, at least one pharmaceutically acceptable salt and/or at least one prodrug thereof to a subject in need thereof.

### BRIEF DESCRIPTION OF THE DRAWINGS

FIG. 1 shows the paw withdrawal threshold obtained with compound 1 in Chung (PWT) at 3, 10 and 30 mg/kg po in saline, 2 ml/kg, administered 1 hour prior to the tactile allodynic test.

FIG. 2 shows tactile thresholds (expressed as % from maximal possible effect) measured three hours after the administration of compound 1 at 6, 20 and 60 mg/kg po in saline (N=6 per data point).

Compounds of formula I and methods for preparing them are known and are, for example, described in PCT/EP 2008/066931, PCT/EP 2008/066934 and PCT/EP 2008/066935. These documents, however, do not describe that the compounds may be useful for treating or preventing pain.

The pharmaceutically acceptable salts of compounds of the formula I, which are also referred to as physiologically tolerated salts, are ordinarily obtainable by reacting the free base of the compounds I of the invention (i.e. of the compounds I according to structural formula I) with suitable acids. Examples of suitable acids are listed in “Fortschritte der Arzneimittelforschung”, 1966, Birkhäuser Verlag, vol. 10, pp. 224-285. These include for example hydrochloric acid, citric acid, tartaric acid, lactic acid, phosphoric acid, methanesulfonic acid, acetic acid, formic acid, maleic acid and fumaric acid.

The term “prodrugs” means compounds which are metabolized in vivo to the compounds I of the invention. Typical examples of prodrugs are described in C. G. Wermeth (editor): The Practice of Medicinal Chemistry, Academic Press, San Diego, 1996, pages 671-715. These include for example phosphates, carbamates, amino acids, esters, amides, peptides, ureas and the like. Suitable prodrugs in the present case may be for example compounds I in which the outer nitrogen atom of the outer nitrogen-containing ring forms an amide/peptide linkage by this nitrogen atom being substituted by a C₁-C₄-alkylcarbonyl group, e.g. by acetyl, propionyl, n-propylcarbonyl, isopropylcarbonyl, n-butylcarbonyl or tert-butylcarbonyl (pivaloyl), by benzoyl, or by an amino acid residue linked via CO, e.g. glycine, alanine, serine, phenylalanine and the like linked via CO, in the position of the radical R³. Further suitable prodrugs are alkylcarbonyloxyalkyl carbamates in which the outer nitrogen atom of the outer nitrogen-containing ring has in the position of the radical R³ a group of the formula —C(═O)—O—CHR^{a}—O—C(═O)—R^{b} in which R^{a} and R^{b} are independently of one another C₁-C₄-alkyl. Such carbamates are described for example in J. Alexander, R. Cargill, S. R. Michelson, H. Schwam, J. Medicinal Chem. 1988, 31(2), 318-322. These groups can then be eliminated under metabolic conditions and result in compounds I in which R³ is H.

C₁-C₃-Alkyl is in the context of the present invention a linear or branched alkyl radical having 1 to 3 carbon atoms, such as methyl, ethyl, n-propyl or isopropyl.

C₁-C₄-Alkyl is in the context of the present invention a linear or branched alkyl radical having 1 to 4 carbon atoms, such as methyl, ethyl, n-propyl, isopropyl, n-butyl, sec-butyl, isobutyl or tert-butyl.

C₁-C₃-Fluoroalkyl is in the context of the present invention a linear or branched alkyl radical having 1 to 3 carbon atoms as defined above, in which at least one hydrogen atom, e.g. 1, 2, 3, 4 or 5 hydrogen atoms, are replaced by fluorine atoms. Example thereof are fluoromethyl, difluoromethyl, trifluoromethyl, 1- and 2-fluoroethyl, 1,1-, 1,2- and 2,2-difluoroethyl, 1,1,2-, 1,2,2 and 2,2,2-trifluoroethyl, 1,1,2,2-tetrafluoroethyl, 1,2,2,2-tetrafluoroethyl, pentafluoroethyl, 1-, 2- and 3-fluoroprop-1-yl, 1,1-, 1,2-, 1,3-, 2,2-, 2,3- and 3,3-difluoroprop-1-yl, 1,1,2-, 1,2,2-, 1,1,3-, 2,2,3-, 1,2,3- and 3,3,3-trifluoroprop-1-yl, 1- and 2-fluoroprop-2-yl, 1,1- and 1,3-difluoroprop-2-yl, 1,1,1-trifluoroprop-2-yl and the like.

C₁-C₃-Alkoxy is in the context of the present invention a linear or branched alkyl radical linked via an oxygen atom and having 1 to 3 carbon atoms. Examples are methoxy, ethoxy, n-propoxy and isopropoxy.

C₁-C₃-Fluoroalkoxy is in the context of the present invention a linear or branched alkyl radical linked via an oxygen atom and having 1 to 3 carbon atoms as defined above, in which at least one hydrogen atom, e.g. 1, 2, 3, 4 or 5 hydrogen atoms, are replaced by fluorine atoms. Example thereof are fluoromethoxy, difluoromethoxy, trifluoromethoxy, 1- and 2-fluoroethoxy, 1,1-, 1,2- and 2,2-difluoroethoxy, 1,1,2-, 1,2,2 and 2,2,2-trifluoroethoxy, 1,1,2,2-tetrafluoroethoxy, 1,2,2,2-tetrafluoroethoxy, pentafluoroethoxy, 1-, 2- and 3-fluoroprop-1-oxy, 1,1-, 1,2-, 1,3-, 2,2-, 2,3- and 3,3-difluoroprop-1-oxy, 1,1,2-, 1,2,2-, 1,1,3-, 2,2,3-, 1,2,3- and 3,3,3-trifluoroprop-1-oxy, 1- and 2-fluoroprop-2-oxy, 1,1- and 1,3-difluoroprop-2-oxy, 1,1,1-trifluoroprop-2-oxy and the like.

Fluorinated ethoxy is in the context of the present invention ethoxy in which 1, 2, 3, 4 or 5 of the hydrogen atoms are replaced by fluorine atoms. Examples are 1-fluoroethoxy, 2-fluoroethoxy, 1,1-difluoroethoxy, 1,2-difluoroethoxy, 2,2-difluoroethoxy, 1,1,2-trifluoroethoxy, 1,2,2-trifluoroethoxy, 2,2,2-trifluoroethoxy, 1,1,2,2-tetrafluoroethoxy, 1,2,2,2-tetrafluoroethoxy and 1,1,2,2,2-pentafluoroethoxy.

Halogen is in the context of the present invention fluorine, chlorine, bromine or iodine.

The compounds of the formula I, their pharmacologically acceptable salts and their prodrugs may also be present in the form of solvates or hydrates. Solvates mean in the context of the present invention crystalline forms of the compounds I or of their pharmaceutically acceptable salts or prodrugs thereof which comprise solvent molecules incorporated in the crystal lattice. The solvent molecules are preferably incorporated in stoichiometric ratios. Hydrates are a specific form of solvates; the solvent in this case is water.

The statements made hereinafter concerning suitable and preferred features of the invention, especially concerning the variables R¹, R², R³, R⁴, R⁵, R⁶, R⁷, R⁸, R⁹, X¹, X², X³, X⁴, a, b, m, n, o and p in the compound I, but also concerning the features of the use and the method according to the invention apply both taken on their own and preferably in any possible combination with one another.

As already stated, the invention preferably relates to the use of compounds of formula I for preparing a medicament for the treatment or prophylaxis of chronic pain.

Chronic pain may be a complex regional pain syndrome, pain arising from peripheral neuropathies, post-operative pain, chronic fatigue syndrome pain, tension-type headache, pain arising from mechanical nerve injury and severe pain associated with diseases such as cancer, metabolic disease, neurotropic viral disease, neurotoxicity, inflammation, multiple sclerosis or any pain arising as a consequence of or associated with stress or depressive illness.

In particular, the invention relates to the use of compounds of formula I or of a pharmaceutically acceptable salt thereof or of a prodrug thereof for preparing a medicament for the treatment or prophylaxis of neuropathic pain.

For diagnostic criteria for chronic pain reference is made to the DSM IV revised edition.

The compounds I are preferably provided in the form of the free base (i.e. according to structural formula I) or in the form of their acid addition salts.

The compounds I have a center of chirality in position 3 of the 2-oxindole ring. The compounds I may therefore be in the form of a 1:1 mixture of enantiomers (racemate) or of a nonracemic mixture of enantiomers in which one of the two enantiomers, either the enantiomer which rotates the plane of vibration of linearly polarized light to the left (i.e. minus rotation) (hereinafter (−) enantiomer) or the enantiomer which rotates the plane of vibration of linearly polarized light to the right (i.e. plus rotation) (hereinafter (+) enantiomer), is enriched, or of substantially enantiopure compounds, that is to say of substantially enantiopure (−) enantiomer or (+) enantiomer. Since the compounds I have a single center of asymmetry and no axis/plane of chirality, a nonracemic mixture can also be defined as a mixture of enantiomers in which either the R or the S enantiomer predominates. Substantially enantiopure compounds can accordingly also be defined as substantially enantiopure R enantiomer or substantially enantiopure S enantiomer.

“Substantially enantiopure compounds” means in the context of the present invention those compounds having an enantiomeric excess (ee; % ee=(R−S)/(R+S)×100 or (S−R)/(S+R)×100) of at least 80% ee, preferably at least 85% ee, more preferably at least 90% ee, even more preferably at least 95% ee and in particular at least 98% ee.

In one embodiment of the invention, the compounds I are in the form of substantially enantiopure compounds. Particularly preferred compounds I have an enantiomeric excess of at least 85% ee, more preferably of at least 90% ee, even more preferably of at least 95% ee and in particular of at least 98% ee.

The invention thus relates to the use of both to the pure enantiomers and to mixtures thereof, e.g. mixtures in which one enantiomer is present in enriched form, but also to the racemates. The invention also relates to the use of the pharmaceutically acceptable salts and the prodrugs of the pure enantiomers of compounds I, and the racemic and nonracemic mixtures of enantiomers in the form of the pharmaceutically acceptable salts and prodrugs of compounds I.

The statements made in the context of the present invention concerning the direction of rotation of polarized light relate preferably to the signs [(+) or (−)] as determined in chloroform as solvent or in chloroform-containing solvent mixtures, in particular in chloroform.

In a preferred embodiment, R¹ and R² are independently of one another hydrogen, C₁-C₃-alkoxy or C₁-C₃-fluoroalkoxy. In this connection, C₁-C₃-alkoxy in the definition of the radicals R¹ and R² is preferably ethoxy or methoxy and is specifically methoxy. C₁-C₃-Fluoroalkoxy is preferably C₁-C₂-fluoroalkoxy, i.e. is fluoromethoxy, difluoromethoxy, trifluoromethoxy, 1- and 2-fluoroethoxy, 1,1-, 1,2- and 2,2-difluoroethoxy, 1,1,2-, 1,2,2 and 2,2,2-trifluoroethoxy, 1,1,2,2-tetrafluoroethoxy, 1,2,2,2-tetrafluoroethoxy, pentafluoroethoxy, is preferably fluoromethoxy, difluoromethoxy, trifluoromethoxy, 2,2-difluoroethoxy and 2,2,2-trifluoroethoxy, and is specifically trifluoromethoxy.

In a preferred embodiment, R¹ is hydrogen, methoxy, ethoxy, fluoromethoxy, difluoromethoxy or trifluoromethoxy, is particularly preferably hydrogen, methoxy or trifluoromethoxy, is more preferably hydrogen or methoxy and is specifically methoxy.

In a preferred embodiment, R² is hydrogen or methoxy and is specifically methoxy.

In a particularly preferred embodiment, at least one of the radicals R¹ and R² is methoxy.

Specifically, both R¹ and R² are methoxy.

In a preferred embodiment, R³ is hydrogen, methyl, ethyl, n-propyl or isopropyl. More preferably, R³ is hydrogen, methyl or ethyl and in particular methyl or ethyl. Specifically, R³ is methyl.

In one embodiment, R⁴ is ethoxy, fluorinated ethoxy or isopropoxy.

In a preferred embodiment, R⁴ is ethoxy and R⁵ is H. In this case, X⁴ is N or CH and is preferably N.

In an alternatively preferred embodiment, R⁴ is ethoxy and R⁵ is methyl. In this case, X⁴ is preferably N.

In an alternatively preferred embodiment, R⁴ is isopropoxy and R⁵ is H. In this case, X⁴ is preferably N.

In an alternatively preferred embodiment, R⁴ is fluorinated ethoxy, is preferably 2,2-difluoroethoxy or 2,2,2-trifluoroethoxy and is particularly preferably 2,2-difluoroethoxy, and R⁵ is H. In this case, X⁴ is N or CH and is specifically CH.

In an alternatively preferred embodiment, R⁴ is methoxy and R⁵ is H. In this case, X⁴ is N or CH and is preferably N.

X⁴ is particularly preferably N.

R⁴ is particularly preferably ethoxy and R⁵ is H. In this case, X⁴ is N or CH and is preferably N.

In a preferred embodiment, R⁶ and R⁷ are not simultaneously CN.

In one embodiment (embodiment A), at least one of the radicals R⁶ and R⁷ is preferably fluorine. Particularly preferably in this case R⁷ is fluorine and R⁶ is fluorine, chlorine, bromine or CN, preferably fluorine, chlorine or CN and more preferably Cl or CN.

In an alternative embodiment (embodiment B), R⁶ is fluorine or chlorine and R⁷ is hydrogen.

In yet another alternative embodiment (embodiment C), R⁶ is CN and R⁷ is hydrogen.

In a preferred embodiment, R⁸ and R⁹ are methyl or ethyl.

In a preferred embodiment, X¹ is NH.

In an alternatively preferred embodiment, X¹ is O.

In an alternatively preferred embodiment, X¹ is CH₂.

X¹ is particularly preferably NH or CH₂ and especially NH.

In a preferred embodiment, one of the variables X², X³ is N and the other is CH.

In a particularly preferred embodiment in this connection, X² is N and X³ is CH.

In an alternatively particularly preferred embodiment, X² is CH and X³ is N.

In an alternatively preferred embodiment, both variables X², X³ are CH. However, it is more preferred that one of X² and X³ is N and the other is CH.

In a preferred embodiment, a and b are independently of one another 0 or 1 and especially 0.

If a and/or b are not equal to 0, it is self-evident that the radicals R⁸ and/or R⁹ are bonded to one of m, n, o or p CH₂ groups, where they replace in each case one hydrogen atom of this CH₂ group.

In a preferred embodiment, m, n, o and p are independently of one another 1 or 2.

Accordingly, m and n are preferably 1 or m and n are 2 or m is 1 and n is 2 or m is 2 and n is 1. It is particularly preferred for m and n to be 2.

Accordingly, o and p are preferably 1 or o and p are 2 or o is 1 and p is 2 or o is 2 and p is 1. It is particularly preferred for o and p to be 2.

In a particularly preferred embodiment, the present invention relates to the use of compounds of the formula I.A

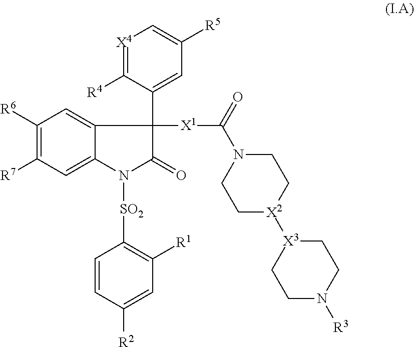

in which R¹, R², R³, R⁴, R⁵, R⁶, R⁷, X¹, X², X³ and X⁴ have the general meanings indicated previously or in particular the preferred meanings indicated previously.

### Embodiments A.1

The invention preferably relates to the use of compounds of the formula I.A in which
R¹ is hydrogen, methoxy or trifluoromethoxy, preferably hydrogen or methoxy, more preferably methoxy;
R² is hydrogen or methoxy, preferably methoxy;
R³ is hydrogen, methyl, ethyl, n-propyl or isopropyl, preferably hydrogen, methyl or ethyl, and in particular methyl or ethyl;
R⁴ is ethoxy;
R⁵ is hydrogen;
R⁶ is Cl, F or CN, preferably Cl or CN;
R⁷ is F or Cl, preferably F;
X¹ is NH, O or CH₂;
X² is N or CH;
X³ is N or CH;
X⁴ is N,
where X² and X³ are not simultaneously N;
   and the pharmaceutically acceptable salts and prodrugs thereof.

The invention preferably relates alternatively to the use of compounds of the formula I.A in which
R¹ is hydrogen, methoxy or trifluoromethoxy, preferably hydrogen or methoxy, more preferably methoxy;
R² is hydrogen or methoxy, preferably methoxy;
R³ is hydrogen, methyl, ethyl, n-propyl or isopropyl, preferably hydrogen, methyl or ethyl, and in particular methyl or ethyl;
R⁴ is 2,2-difluoroethoxy or ethoxy;
R⁵ is hydrogen;
R⁶ is Cl, F or CN, preferably Cl or CN;
R⁷ is F or Cl, preferably F;
X¹ is NH, O or CH₂;
X² is N or CH;
X³ is N or CH;
X⁴ is CH
where X² and X³ are not simultaneously N;
   and the pharmaceutically acceptable salts and prodrugs thereof.

The invention particularly preferably relates to the use of compounds of the formula I.A in which
R¹ is hydrogen or methoxy, preferably methoxy;
R² is hydrogen or methoxy, preferably methoxy;
R³ is methyl or ethyl;
R⁴ is ethoxy;
R⁵ is hydrogen;
R⁶ is Cl, F or CN, preferably Cl or CN;
R⁷ is F or Cl, preferably F;
X¹ is NH, O or CH₂;
X² is N or CH;
X³ is N or CH;
X⁴ is N;
where X² and X³ are not simultaneously N;
   and the pharmaceutically acceptable salts and prodrugs thereof.

The invention particularly preferably relates alternatively to the use of compounds of the formula I.A in which
R¹ is hydrogen or methoxy, preferably methoxy;
R² is hydrogen or methoxy, preferably methoxy;
R³ is methyl or ethyl;
R⁴ is 2,2-difluoroethoxy or ethoxy;
R⁵ is hydrogen;
R⁶ is Cl, F or CN, preferably Cl or CN;
R⁷ is F or Cl, preferably F;
X¹ is NH, O or CH₂;
X² is N or CH;
X³ is N or CH;
X⁴ is CH;
where X² and X³ are not simultaneously N;
   and the pharmaceutically acceptable salts and prodrugs thereof.

The invention more preferably relates to the use of compounds of the formula I.A in which
R¹ is methoxy or H, preferably methoxy;
R² is methoxy;
R³ is methyl or ethyl;
R⁴ is ethoxy;
R⁵ is hydrogen;
R⁶ is Cl, F or CN, preferably Cl or CN;
R⁷ is F;
X¹ is NH, O or CH₂;
X² is N or CH;
X³ is N or CH;
X⁴ is N;
where X² and X³ are not simultaneously N;
   and the pharmaceutically acceptable salts and prodrugs thereof.

The invention more preferably relates alternatively to the use of compounds of the formula I.A in which
R¹ is methoxy or H, preferably methoxy;
R² is methoxy;
R³ is methyl or ethyl;
R⁴ is ethoxy;
R⁵ is hydrogen;
R⁶ is Cl, F or CN, preferably Cl or CN;
R⁷ is F;
X¹ is NH, O or CH₂;
X² is N or CH;
X³ is N or CH;
X⁴ is CH;
where X² and X³ are not simultaneously N;
   and the pharmaceutically acceptable salts and prodrugs thereof.

The invention even more preferably relates to the use of compounds of the formula I.A in which
R¹ is methoxy;
R² is methoxy;
R³ is methyl or ethyl;
R⁴ is ethoxy;
R⁵ is hydrogen;
R⁶ is Cl or CN;
R⁷ is F;
X¹ is NH;
X² is N;
X³ is CH;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is Cl or CN;
R⁷ is F;
X¹ is NH;
X² is CH;
X³ is N;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is NH;
X² is N;
X³ is CH;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is NH;
X² is CH;
X³ is N;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is Cl or CN;
R⁷ is F;
X¹ is CH₂;
X² is N;
X³ is CH;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is Cl or CN;
R⁷ is F;
X¹ is CH₂;
X² is CH;
X³ is N;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is CH₂;
X² is N;
X³ is CH;
X⁴ is N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is CH₂;
X² is CH;
X³ is N;
X⁴ is N;
   and the pharmaceutically acceptable salts and prodrugs thereof.

The invention even more preferably relates alternatively to the use of compounds of the formula I.A in which
R¹ is methoxy or hydrogen;
R² is methoxy;
R³ is methyl or ethyl;
R⁴ is ethoxy;
R⁵ is hydrogen;
R⁶ is Cl or CN;
R⁷ is F;
X¹ is O;
X² is N;
X³ is CH;
X⁴ is N;
   and the pharmaceutically acceptable salts and prodrugs thereof.

The invention even more preferably relates alternatively to the use of compounds of the formula I.A in which
R¹ is methoxy or hydrogen;
R² is methoxy;
R³ is methyl or ethyl;
R⁴ is ethoxy;
R⁵ is hydrogen;
R⁶ is Cl or CN;
R⁷ is F;
X¹ is O;
X² is CH;
X³ is N;
X⁴ is N;
   and the pharmaceutically acceptable salts and prodrugs thereof.

The invention even more preferably relates alternatively to the use of compounds of the formula I.A in which
R¹ is methoxy or hydrogen;
R² is methoxy;
R³ is methyl or ethyl;
R⁴ is ethoxy;
R⁵ is hydrogen;
R⁶ is F;
R⁷ is F;
X¹ is O;
X² is N;
X³ is CH;
X⁴ is N;
   and the pharmaceutically acceptable salts and prodrugs thereof.

The invention even more preferably relates alternatively to the use of compounds of the formula I.A in which
R¹ is methoxy or hydrogen;
R² is methoxy;
R³ is methyl or ethyl;
R⁴ is ethoxy;
R⁵ is hydrogen;
R⁶ is F;
R⁷ is F;
X¹ is O;
X² is CH;
X³ is N;
X⁴ is N;
   and the pharmaceutically acceptable salts and prodrugs thereof.

The invention even more preferably relates alternatively to the use of compounds of the formula I.A in which
R¹ is methoxy or hydrogen;
R² is methoxy;
R³ is methyl or ethyl;
R⁴ is ethoxy;
R⁵ is hydrogen;
R⁶ is Cl or CN;
R⁷ is F;
X¹ is NH;
X² is N;
X³ is CH;
X⁴ is CH;
   and the pharmaceutically acceptable salts and prodrugs thereof.

The invention even more preferably relates alternatively to the use of compounds of the formula I.A in which
R¹ is methoxy or hydrogen;
R² is methoxy;
R³ is methyl or ethyl;
R⁴ is ethoxy;
R⁵ is hydrogen;
R⁶ is Cl or CN;
R⁷ is F;
X¹ is NH;
X² is CH;
X³ is N;
X⁴ is CH;
   and the pharmaceutically acceptable salts and prodrugs thereof.

The invention particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is F;
X¹ is NH;
X² is N;
X³ is CH;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is F;
X¹ is NH;
X² is CH;
X³ is N;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is F;
X¹ is CH₂;
X² is N;
X³ is CH;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is F;
X¹ is CH₂;
X² is CH;
X³ is N;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is CN;
R⁷ is F;
X¹ is NH;
X² is N;
X³ is CH;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is CN;
R⁷ is F;
X¹ is NH;
X² is CH;
X³ is N;
X⁴ is N;
   and the pharmaceutically acceptable salts and prodrugs thereof.

The invention also particularly relates to the use of compounds of the formula I.A in which
R¹ is methoxy or hydrogen;
R² is methoxy;
R³ is methyl or ethyl;
R⁴ is ethoxy;
R⁵ is hydrogen;
R⁶ is CN;
R⁷ is F;
X¹ is O;
X² is N;
X³ is CH;
X⁴ is N;
   and the pharmaceutically acceptable salts and prodrugs thereof.

The invention also particularly relates to the use of compounds of the formula I.A in which
R¹ is methoxy or hydrogen;
R² is methoxy;
R³ is methyl or ethyl;
R⁴ is ethoxy;
R⁵ is hydrogen;
R⁶ is CN;
R⁷ is F;
X¹ is O;
X² is CH;
X³ is N;
X⁴ is N;
   and the pharmaceutically acceptable salts and prodrugs thereof.

The invention particularly relates to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is NH;
X² is N;
X³ is CH;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is NH;
X² is CH;
X³ is N;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is CH₂;
X² is N;
X³ is CH;
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is F;
R⁷ is F;
X¹ is CH₂;
X² is CH;
X³ is N;
X⁴ is N;
   and the pharmaceutically acceptable salts and prodrugs thereof.

S

### Embodiment B.1

The invention preferably relates to the use of compounds of the formula I.A in which
R¹ is hydrogen or methoxy, preferably methoxy;
R² is hydrogen or methoxy, preferably methoxy;
R³ is hydrogen, methyl, ethyl, n-propyl or isopropyl, preferably hydrogen, methyl or ethyl;
R⁴ is ethoxy;
R⁵ is hydrogen;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention particularly preferably relates to the use of compounds of the formula I.A in which
R¹ is hydrogen or methoxy, preferably methoxy;
R² is hydrogen or methoxy, preferably methoxy;
R³ is methyl or ethyl;
R⁴ is ethoxy;
R⁵ is hydrogen;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention more preferably relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention even more preferably relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is hydrogen;
X¹ is NH;
X² is N or CH;
X³ is N or CH; and
X⁴ is N;
where X² and X³ are not simultaneously N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is hydrogen;
X¹ is CH₂;
X² is N or CH;
X³ is N or CH; and
X⁴ is N;
where X² and X³ are not simultaneously N;
   and the pharmaceutically acceptable salts and prodrugs thereof.

The invention particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is hydrogen;
X¹ is NH;
X² is N;
X³ is CH; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is hydrogen;
X¹ is NH;
X² is CH;
X³ is N; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is hydrogen;
R⁶ is Cl;
R⁷ is hydrogen;
X¹ is NH;
X² is CH;
X³ is CH; and
X⁴ is N;
   and the pharmaceutically acceptable salts and prodrugs thereof.

The invention preferably relates alternatively to the use of compounds of the formula I.A in which
R¹ is hydrogen or methoxy, preferably methoxy;
R² is hydrogen or methoxy, preferably methoxy;
R³ is hydrogen, methyl, ethyl, n-propyl or isopropyl, preferably hydrogen, methyl or ethyl;
R⁴ is ethoxy;
R⁵ is methyl;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention particularly preferably relates alternatively to the use of compounds of the formula I.A in which
R¹ is hydrogen or methoxy, preferably methoxy;
R² is hydrogen or methoxy, preferably methoxy;
R³ is methyl or ethyl;
R⁴ is ethoxy;
R⁵ is methyl;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention more preferably relates alternatively to the use of compounds of the formula I.A in which
R¹ is methoxy;
R² is methoxy;
R³ is methyl or ethyl;
R⁴ is ethoxy;
R⁵ is methyl;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention more preferably still relates alternatively to the use of compounds of the formula I.A in which
R¹ is methoxy;
R² is methoxy;
R³ is methyl or ethyl;
R⁴ is ethoxy;
R⁵ is methyl;
R⁶ is Cl;
R⁷ is hydrogen;
X¹ is NH;
X² is N or CH;
X³ is N or CH; and
X⁴ is N;
where X² and X³ are not simultaneously N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is ethoxy;
R⁵ is methyl;
R⁶ is Cl;
R⁷ is hydrogen;
X¹ is CH₂;
X² is N or CH;
X³ is N or CH; and
X⁴ is N;
where X² and X³ are not simultaneously N;
   and the pharmaceutically acceptable salts and prodrugs thereof.

The invention particularly relates to the use of compounds of the formula I.A in which
R¹ is methoxy;
R² is methoxy;
R³ is methyl or ethyl;
R⁴ is ethoxy;
R⁵ is methyl;
R⁶ is Cl;
R⁷ is hydrogen;
X¹ is NH;
X² is N;
X³ is CH; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is methyl;
R⁶ is Cl;
R⁷ is hydrogen;
X¹ is NH;
X² is CH;
X³ is N; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is ethoxy;
R⁵ is methyl;
R⁶ is Cl;
R⁷ is hydrogen;
X¹ is NH;
X² is CH;
X³ is CH; and
X⁴ is N;
   and the pharmaceutically acceptable salts and prodrugs thereof.

The invention alternatively preferably relates to the use of compounds of the formula I.A in which
R¹ is hydrogen or methoxy, preferably methoxy;
R² is hydrogen or methoxy, preferably methoxy;
R³ is hydrogen, methyl, ethyl, n-propyl or isopropyl, preferably hydrogen, methyl or ethyl;
R⁴ is methoxy;
R⁵ is hydrogen;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention particularly preferably relates to the use of compounds of the formula I.A in which
R¹ is hydrogen or methoxy, preferably methoxy;
R² is hydrogen or methoxy, preferably methoxy;
R³ is methyl or ethyl;
R⁴ is methoxy;
R⁵ is hydrogen;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention more preferably relates to the use of compounds of the formula I.A in which
R¹ is methoxy;
R² is methoxy;
R³ is methyl or ethyl;
R⁴ is methoxy;
R⁵ is hydrogen;
R⁶ is Cl;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention even more preferably relates to the use of compounds of the formula I.A in which
R¹ is methoxy;
R² is methoxy;
R³ is methyl or ethyl;
R⁴ is methoxy;
R⁵ is hydrogen;
R⁶ is Cl;
R⁷ is hydrogen;
X¹ is NH;
X² is N or CH;
X³ is N or CH; and
X⁴ is N;
where X² and X³ are not simultaneously N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is methoxy;
R⁵ is hydrogen;
R⁶ is Cl;
R⁷ is hydrogen;
X¹ is CH₂;
X² is N or CH;
X³ is N or CH; and
X⁴ is N;
where X² and X³ are not simultaneously N;
   and the pharmaceutically acceptable salts and prodrugs thereof.

The invention particularly relates to the use of compounds of the formula I.A in which
R¹ is methoxy;
R² is methoxy;
R³ is methyl or ethyl;
R⁴ is methoxy;
R⁵ is hydrogen;
R⁶ is Cl;
R⁷ is hydrogen;
X¹ is NH;
X² is N;
X³ is CH; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is methoxy;
R⁵ is hydrogen;
R⁶ is Cl;
R⁷ is hydrogen;
X¹ is NH;
X² is CH;
X³ is N; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is methoxy;
R⁵ is hydrogen;
R⁶ is Cl;
R⁷ is hydrogen;
X¹ is NH;
X² is CH;
X³ is CH; and
X⁴ is N;
   and the pharmaceutically acceptable salts and prodrugs thereof.

Special preference is given to the use of compound I.A in which
R¹ is methoxy;
R² is methoxy;
R³ is methyl;
R⁴ is ethoxy;
R⁵ is hydrogen;
R⁶ is Cl;
R⁷ is hydrogen;
X¹ is NH;
X² is N;
X³ is CH; and
X⁴ is N;
   and the pharmaceutically acceptable salts and prodrugs thereof.

### Embodiment C.1

The invention preferably relates to the use of compounds of the formula I.A in which
R¹ is hydrogen or methoxy, preferably methoxy;
R² is hydrogen or methoxy, preferably methoxy;
R³ is hydrogen, methyl, ethyl, n-propyl or isopropyl, preferably hydrogen, methyl or ethyl;
R⁴ is ethoxy;
R⁵ is hydrogen;
R⁶ is CN;
R⁷ is hydrogen;
X¹ is CH₂;
X² is N or CH;
X³ is N or CH; and
X⁴ is N;
where X¹ and X² are not simultaneously N;
   and the pharmaceutically acceptable salts and prodrugs thereof. In a particular embodiment, the variables X² and X³ are not simultaneously CH, i.e. preferably one of the variables X² or X³ is N and the other is CH.

The invention particularly preferably relates to the use of compounds of the formula I.A in which
R¹ is hydrogen or methoxy, preferably methoxy;
R² is hydrogen or methoxy, preferably methoxy;
R³ is hydrogen, methyl or ethyl;
R⁴ is ethoxy;
R⁵ is hydrogen;
R⁶ is CN;
R⁷ is hydrogen;
X¹ is CH₂;
X² is N;
X³ is CH; and
X⁴ is N;
   and the pharmaceutically acceptable salts and prodrugs thereof.

Among these, preference is given to the use of the compound I.A in which
R¹ is methoxy;
R² is methoxy;
R³ is methyl;
R⁴ is ethoxy;
R⁵ is hydrogen;
R⁶ is CN;
R⁷ is hydrogen;
X¹ is CH₂;
X² is N; and
X³ is CH; and
X⁴ is N;
   and the use of the compound I in which
R¹ is methoxy;
R² is methoxy;
R³ is ethyl;
R⁴ is ethoxy;
R⁵ is hydrogen;
R⁶ is CN;
R⁷ is hydrogen;
X¹ is CH₂;
X² is N; and
X³ is CH; and
X⁴ is N

The invention alternatively particularly preferably relates to the use of compounds of the formula I.A in which
R¹ is hydrogen or methoxy, preferably methoxy;
R² is hydrogen or methoxy, preferably methoxy;
R³ is hydrogen, methyl or ethyl;
R⁴ is ethoxy;
R⁵ is hydrogen;
R⁶ is CN;
R⁷ is hydrogen;
X¹ is CH₂;
X² is CH;
X³ is N; and
X⁴ is N;
   and the pharmaceutically acceptable salts and prodrugs thereof.

Among these, preference is given to the use of the compound I.A in which
R¹ is methoxy;
R² is methoxy;
R³ is methyl;
R⁴ is ethoxy;
R⁵ is hydrogen;
R⁶ is CN;
R⁷ is hydrogen;
X¹ is CH₂;
X² is CH; and
X³ is N; and
X⁴ is N;
   and the use of the compound I in which
R¹ is methoxy;
R² is methoxy;
R³ is ethyl;
R⁴ is ethoxy;
R⁵ is hydrogen;
R⁶ is CN;
R⁷ is hydrogen;
X¹ is CH₂;
X² is CH; and
X³ is N; and
X⁴ is N.

The invention further particularly preferably relates to the use of compounds of the formula I.A in which
R¹ is hydrogen or methoxy, preferably methoxy;
R² is hydrogen or methoxy, preferably methoxy;
R³ is hydrogen, methyl or ethyl;
R⁴ is ethoxy;
R⁵ is hydrogen;
R⁶ is CN;
R⁷ is hydrogen;
X¹ is CH₂;
X² is CH;
X³ is CH; and
X⁴ is N;
   and the pharmaceutically acceptable salts and prodrugs thereof.

Among these, preference is given to the compound I in which
R¹ is methoxy;
R² is methoxy;
R³ is methyl;
R⁴ is ethoxy;
R⁵ is hydrogen;
R⁶ is CN;
R⁷ is hydrogen;
X¹ is CH₂;
X² is CH; and
X³ is CH; and
X⁴ is N;
   and the compound I in which
R¹ is methoxy;
R² is methoxy;
R³ is ethyl;
R⁴ is ethoxy;
R⁵ is hydrogen;
R⁶ is CN;
R⁷ is hydrogen;
X¹ is CH₂;
X² is CH; and
X³ is CH; and
X⁴ is N.

The invention alternatively preferably relates to the use of compounds of the formula I.A in which
R¹ is hydrogen or methoxy, preferably methoxy;
R² is hydrogen or methoxy, preferably methoxy;
R³ is hydrogen, methyl, ethyl, n-propyl or isopropyl, preferably hydrogen, methyl or ethyl;
R⁴ is methoxy;
R⁵ is hydrogen;
R⁶ is CN;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention particularly preferably relates to the use of compounds of the formula I.A in which
R¹ is hydrogen or methoxy, preferably methoxy;
R² is hydrogen or methoxy, preferably methoxy;
R³ is methyl or ethyl;
R⁴ is methoxy;
R⁵ is hydrogen;
R⁶ is CN;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention more preferably relates to the use of compounds of the formula I.A in which
R¹ is methoxy;
R² is methoxy;
R³ is methyl or ethyl;
R⁴ is methoxy;
R⁵ is hydrogen;
R⁶ is CN;
R⁷ is hydrogen;
X¹ is NH, O or CH₂;
X² is N or CH;
X³ is N or CH; and
X⁴ is N;
where X² and X³ are not simultaneously N;
   and the pharmaceutically acceptable salts and prodrugs thereof.

The invention even more preferably relates to the use of compounds of the formula I.A in which
R¹ is methoxy;
R² is methoxy;
R³ is methyl or ethyl;
R⁴ is methoxy;
R⁵ is hydrogen;
R⁶ is CN;
R⁷ is hydrogen;
X¹ is NH;
X² is N or CH;
X³ is N or CH; and
X⁴ is N;
where X² and X³ are not simultaneously N;
   and the pharmaceutically acceptable salts and prodrugs thereof.

The invention even more preferably relates alternatively to the use of compounds of the formula I.A in which
R¹ is methoxy;
R² is methoxy;
R³ is methyl or ethyl;
R⁴ is methoxy;
R⁵ is hydrogen;
R⁶ is CN;
R⁷ is hydrogen;
X¹ is CH₂;
X² is N or CH;
X³ is N or CH; and
X⁴ is N;
where X² and X³ are not simultaneously N;
   and the pharmaceutically acceptable salts and prodrugs thereof.

The invention particularly relates to the use of compounds of the formula I.A in which
R¹ is methoxy;
R² is methoxy;
R³ is methyl or ethyl;
R⁴ is methoxy;
R⁵ is hydrogen;
R⁶ is CN;
R⁷ is hydrogen;
X¹ is NH;
X² is N;
X³ is CH; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is methoxy;
R⁵ is hydrogen;
R⁶ is CN;
R⁷ is hydrogen;
X¹ is NH;
X² is CH;
X³ is N; and
X⁴ is N;
   and the pharmaceutically acceptable salts and prodrugs thereof.

The invention also particularly relates to the use of compounds of the formula I.A in which
R¹ is methoxy;
R² is methoxy;
R³ is methyl or ethyl;
R⁴ is methoxy;
R⁵ is hydrogen;
R⁶ is CN;
R⁷ is hydrogen;
X¹ is NH;
X² is CH;
X³ is CH; and
X⁴ is N;
   and the pharmaceutically acceptable salts and prodrugs thereof.

Examples of preferred embodiment of the present invention are compounds of the formula I.1 to I.85 to be used according to the invention and the pharmaceutically acceptable salts and prodrugs thereof, in which the radicals X², X³, R¹, R² and R³ assume in each case the meanings mentioned in each line in the following table 1.

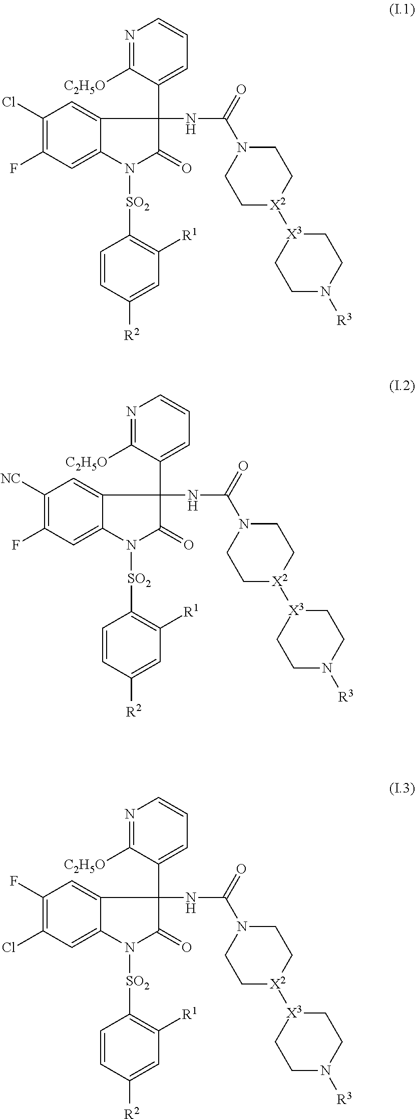

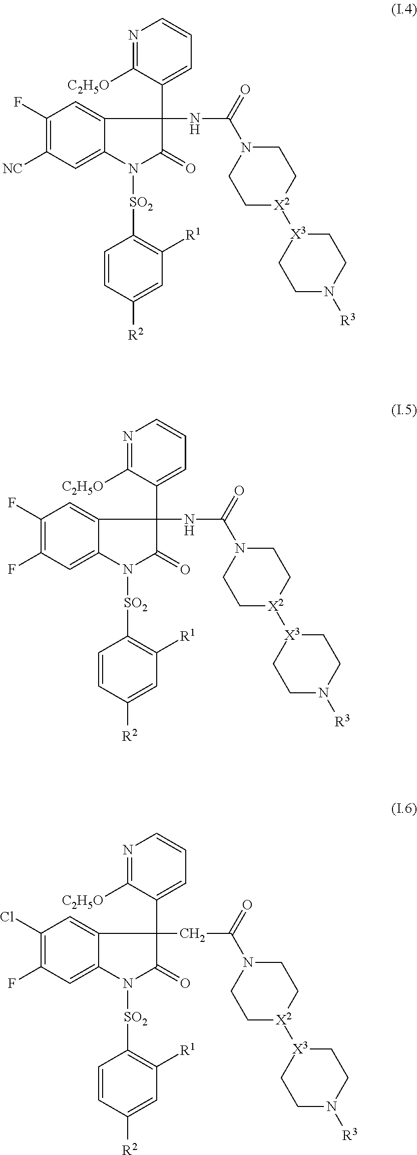

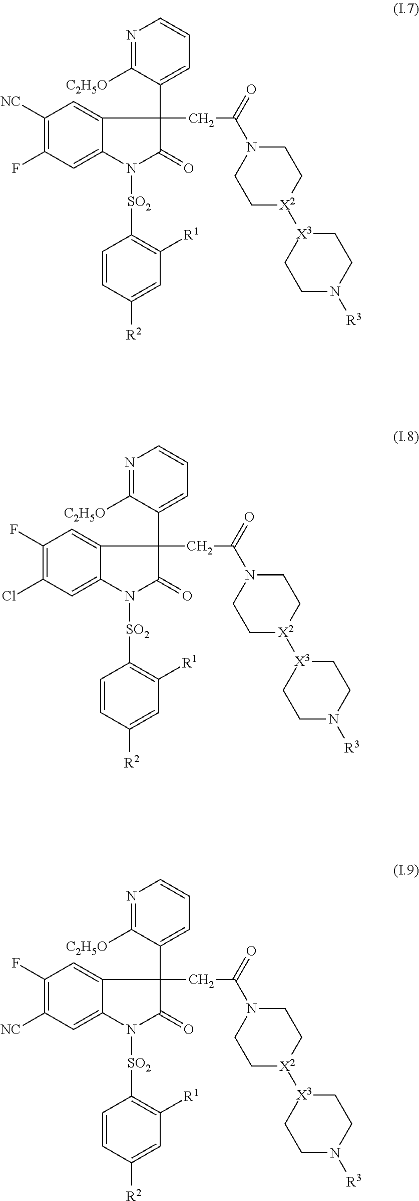

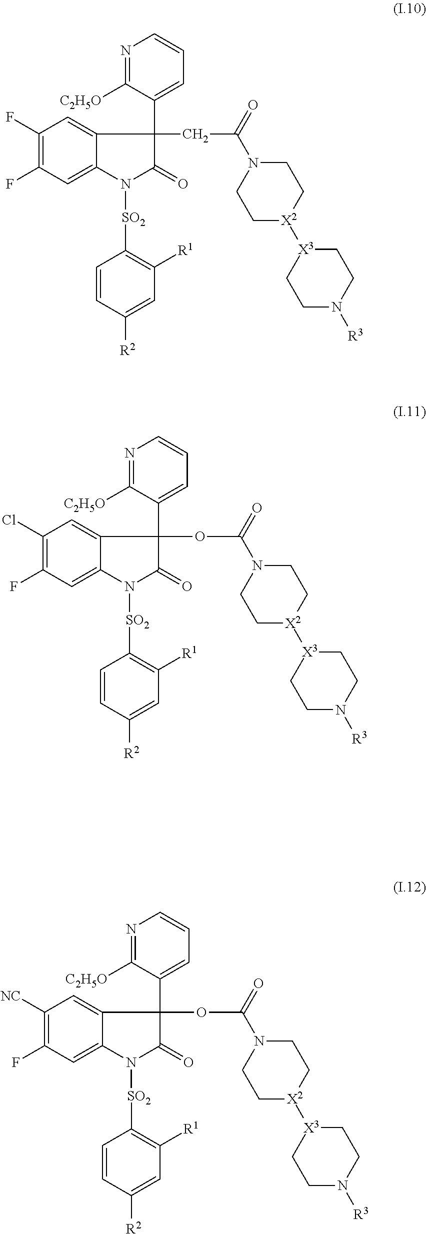

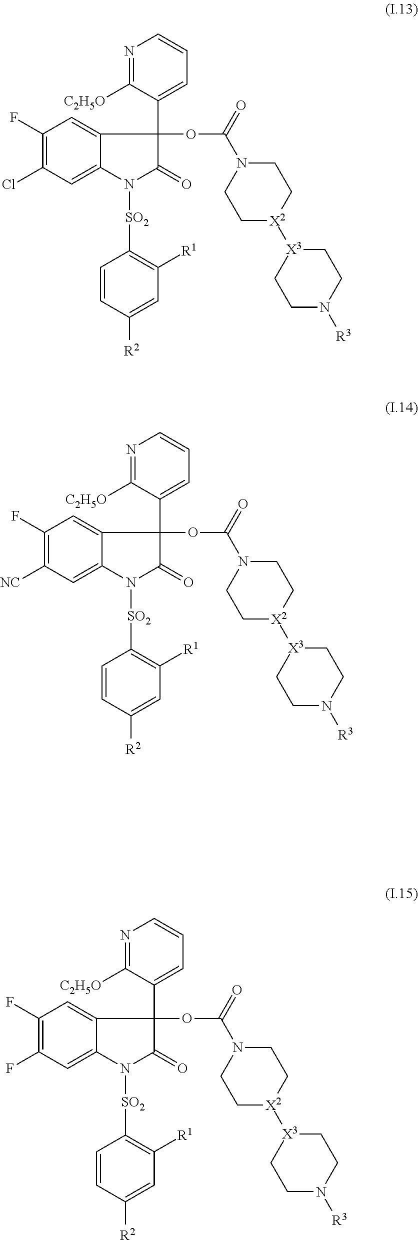

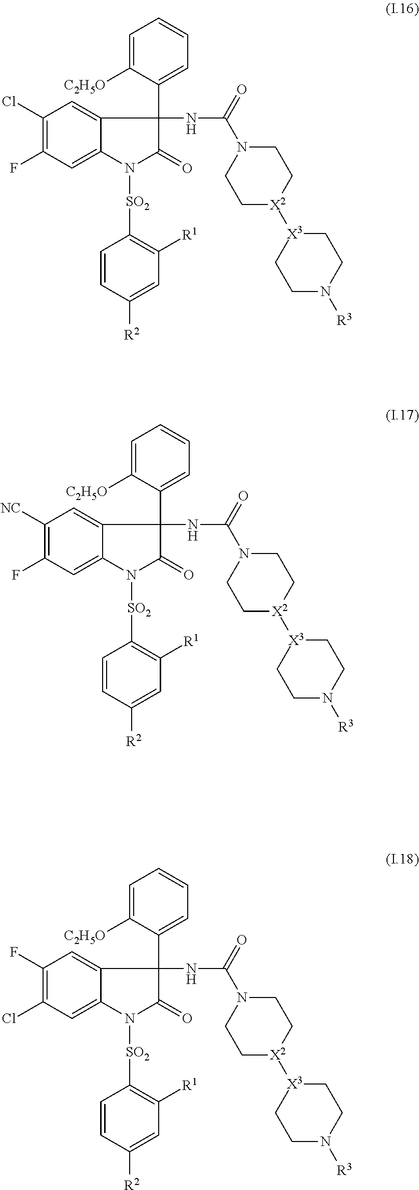

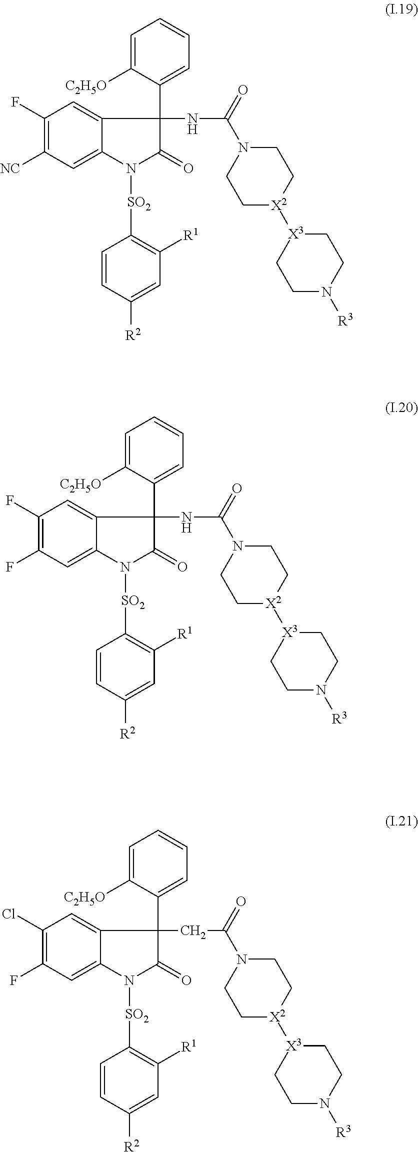

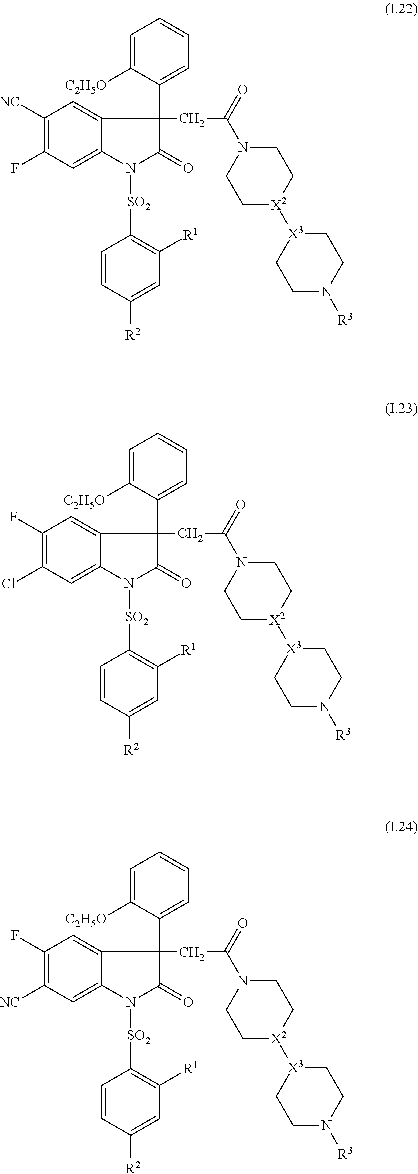

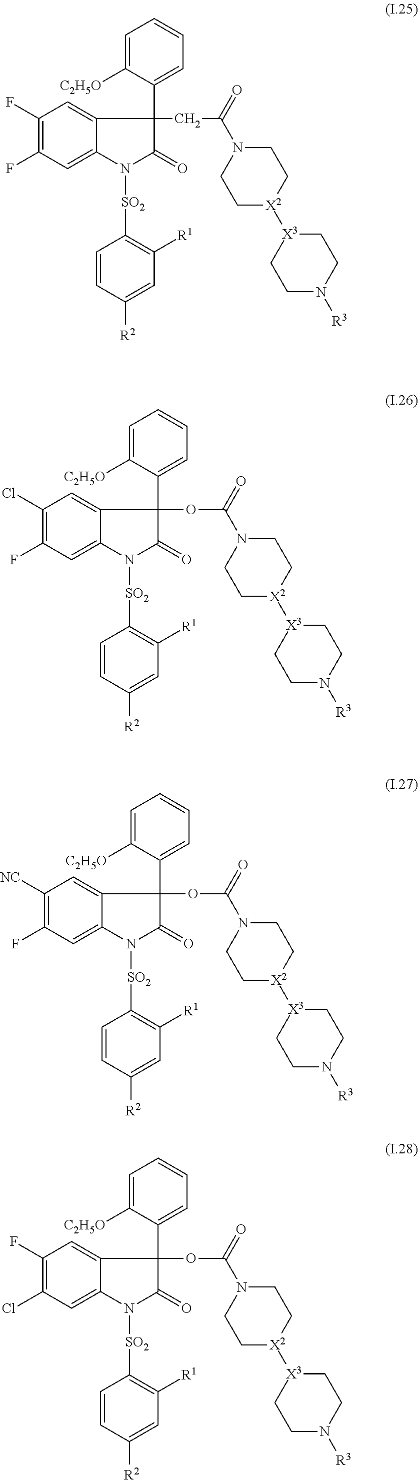

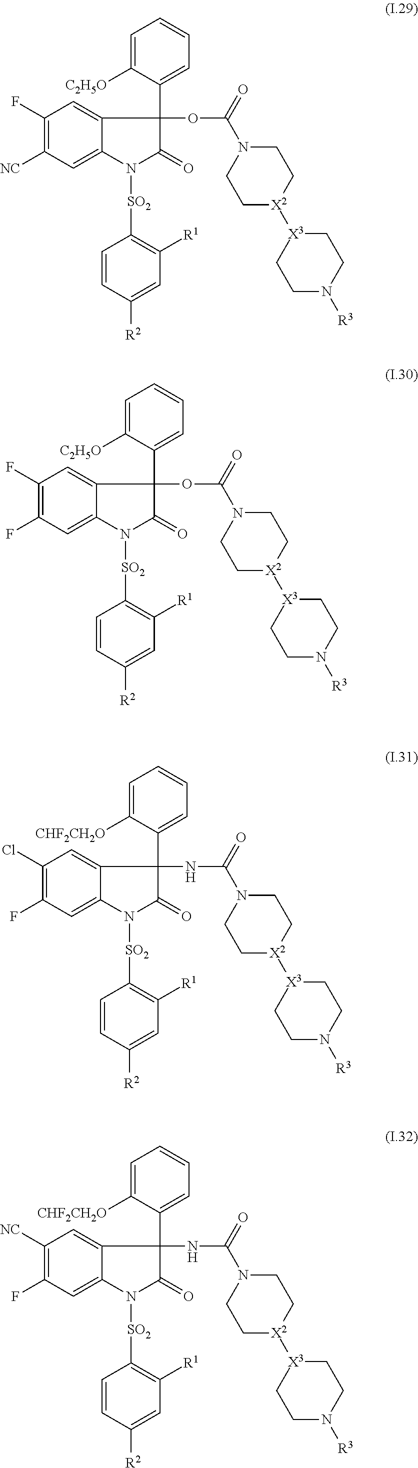

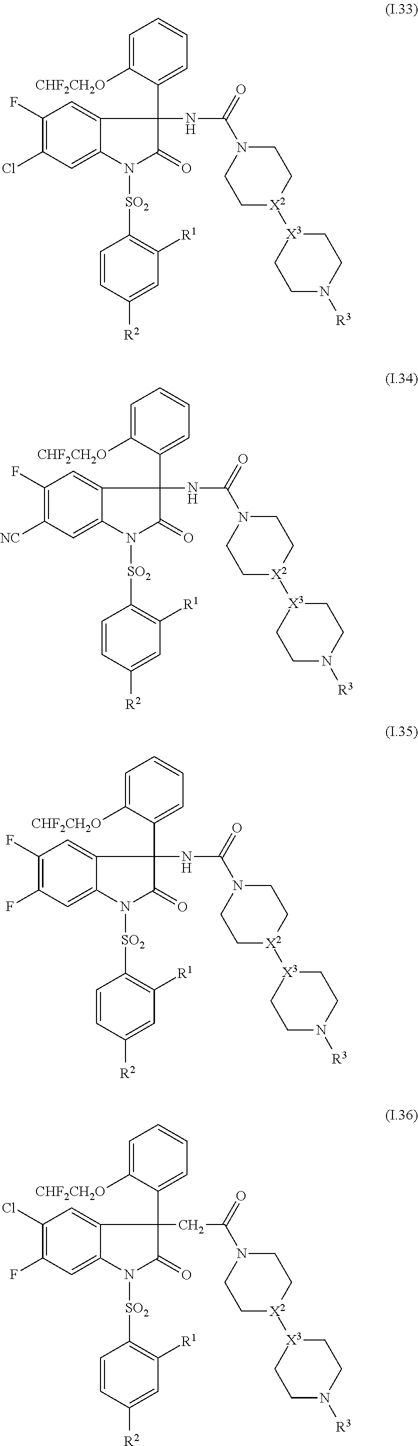

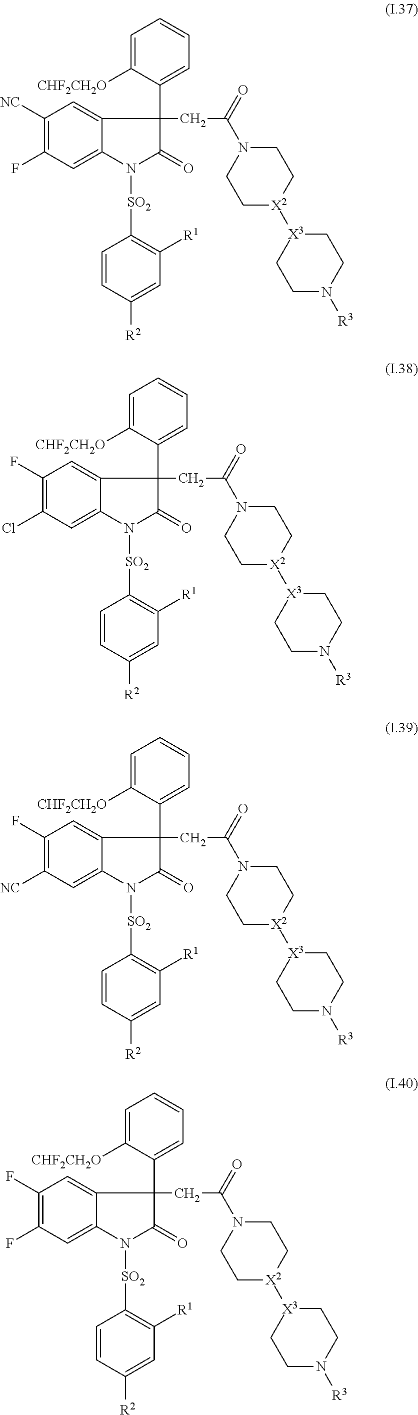

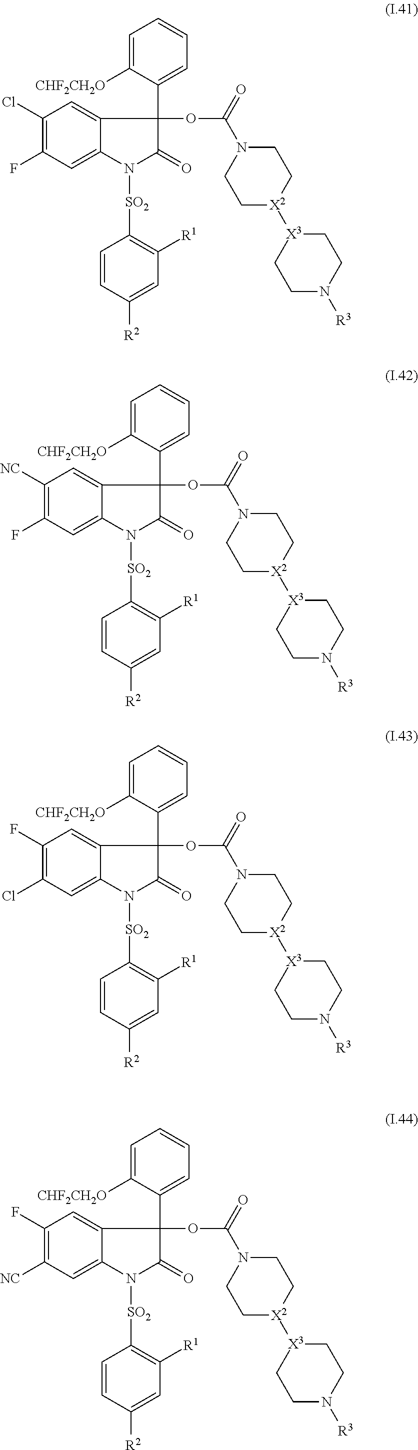

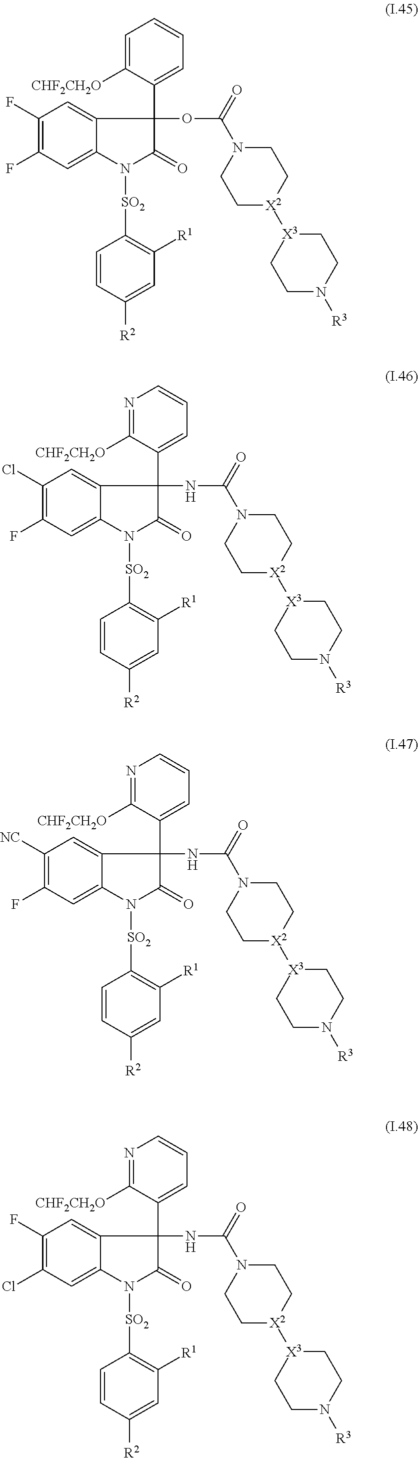

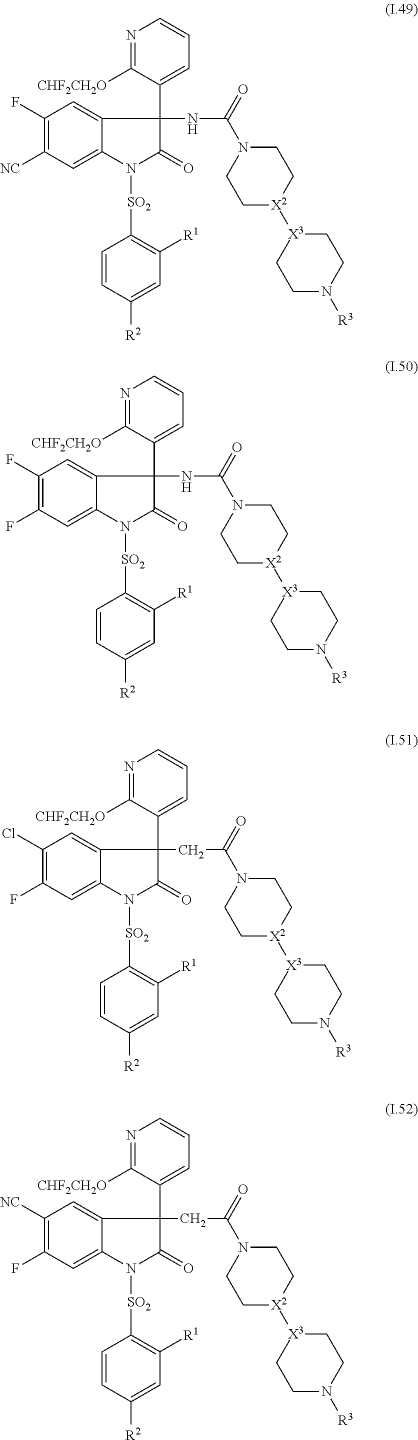

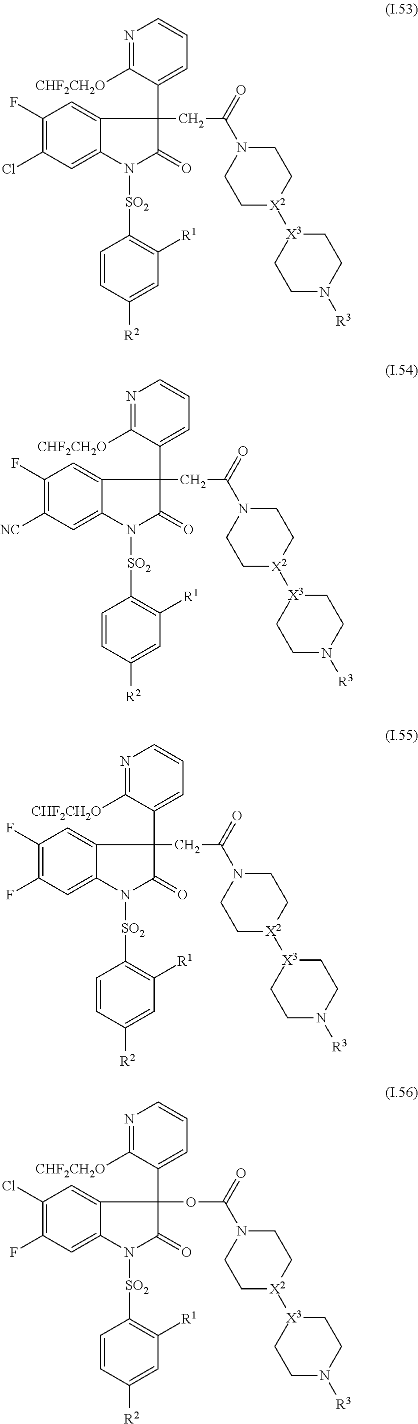

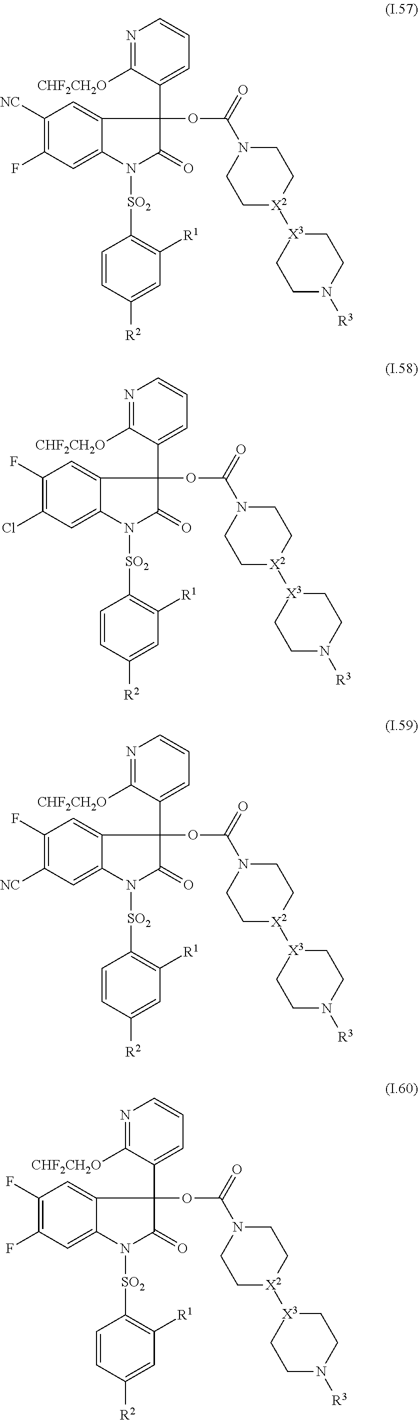

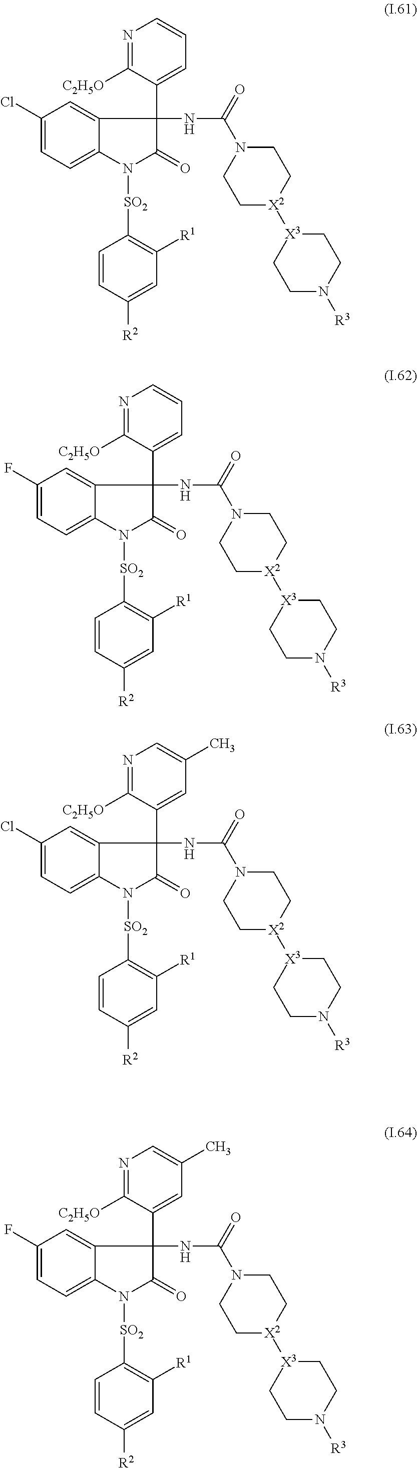

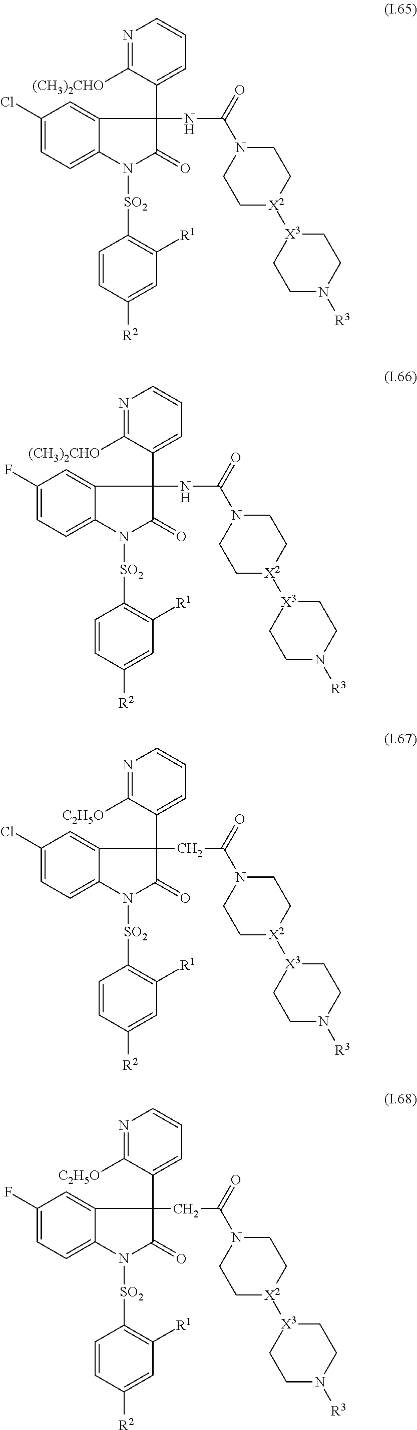

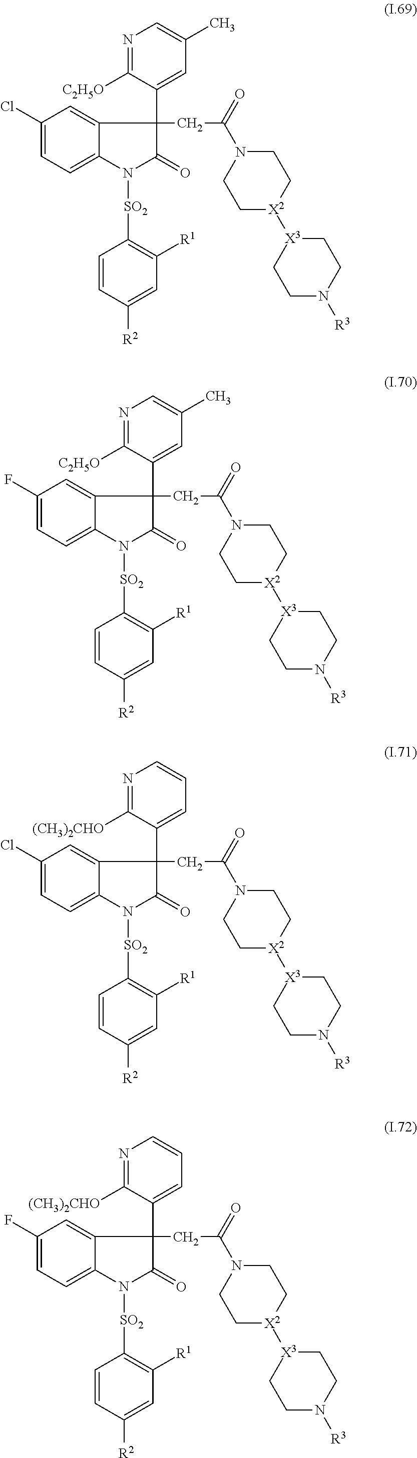

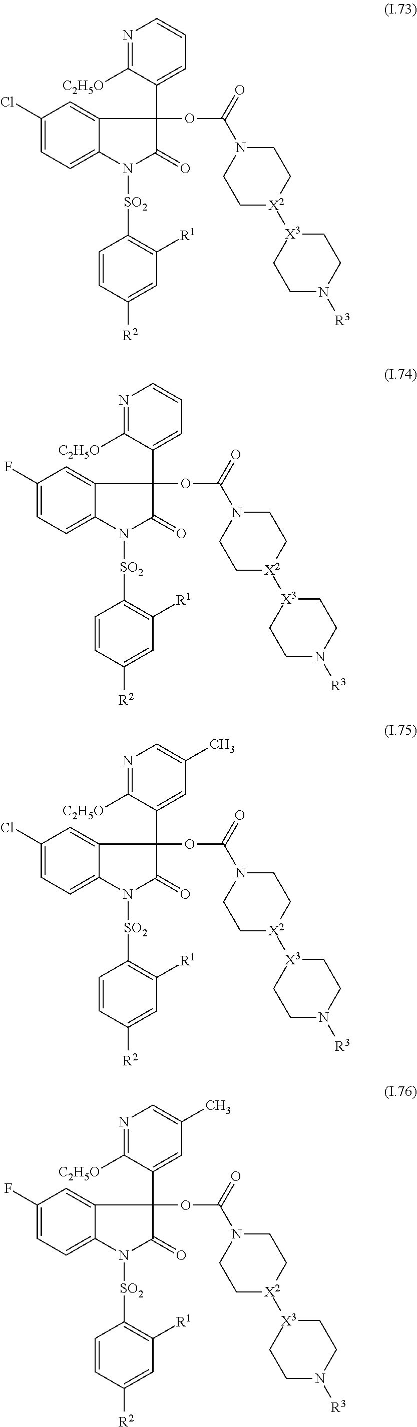

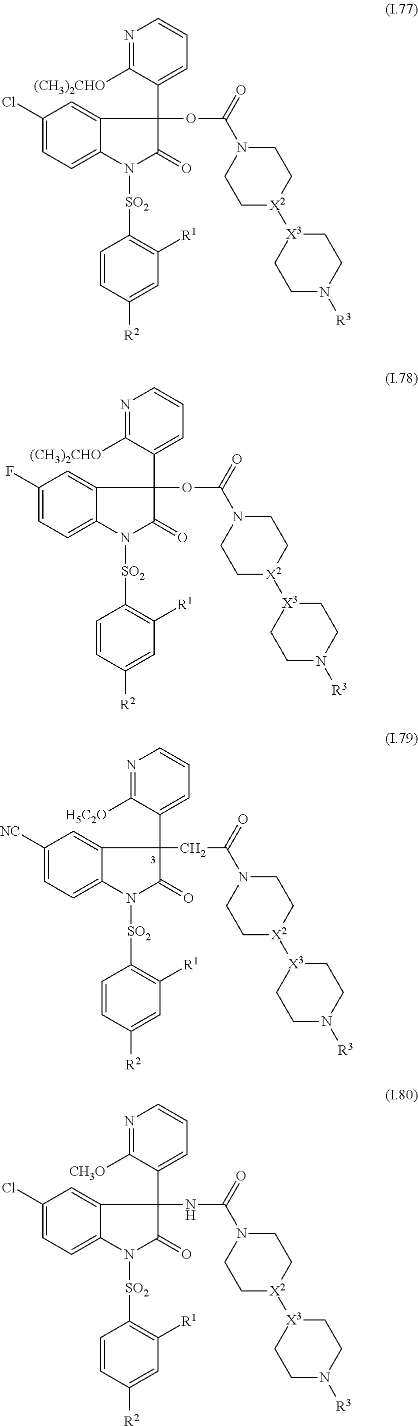

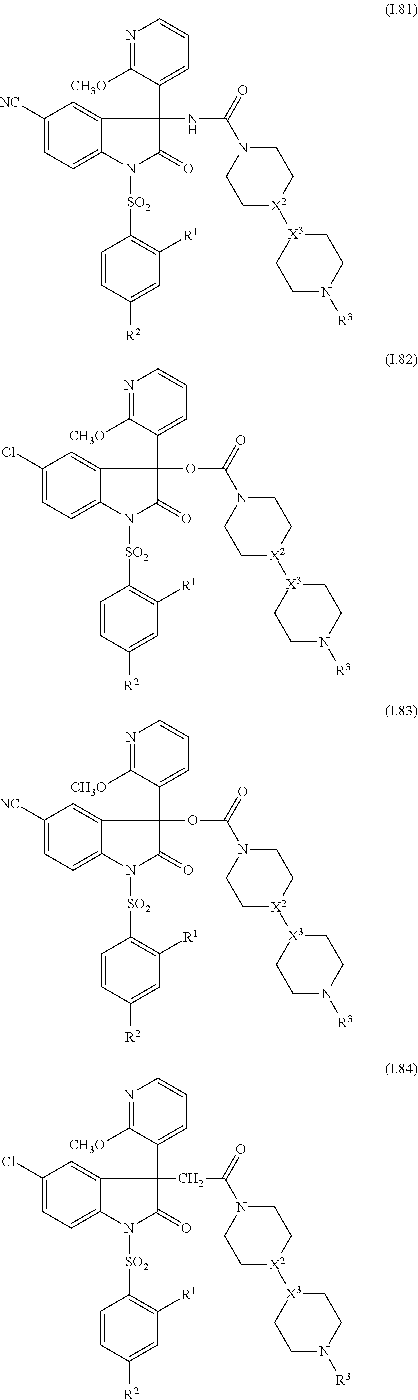

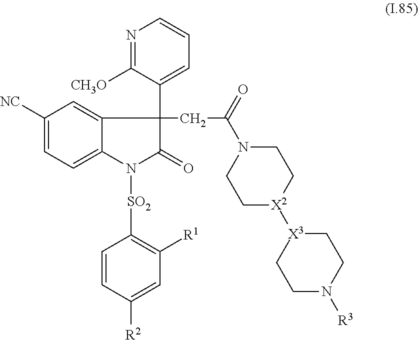

TABLE 1

| Example No. | X² | X³ | R¹ | R² | R³ |
|---|---|---|---|---|---|
| A-1. | N | CH | Methoxy | Methoxy | Methyl |
| A-2. | N | CH | Methoxy | H | Methyl |
| A-3. | N | CH | Ethoxy | H | Methyl |
| A-4. | N | CH | H | H | Methyl |
| A-5. | N | CH | H | Methoxy | Methyl |
| A-6. | N | CH | Ethoxy | Methoxy | Methyl |
| A-7. | N | CH | Methoxy | Methoxy | Ethyl |
| A-8. | N | CH | Methoxy | H | Ethyl |
| A-9. | N | CH | Ethoxy | H | Ethyl |
| A-10. | N | CH | H | H | Ethyl |
| A-11. | N | CH | H | Methoxy | Ethyl |
| A-12. | N | CH | Ethoxy | Methoxy | Ethyl |
| A-13. | N | CH | Methoxy | Methoxy | n-Propyl |
| A-14. | N | CH | Methoxy | H | n-Propyl |
| A-15. | N | CH | Ethoxy | H | n-Propyl |
| A-16. | N | CH | H | H | n-Propyl |
| A-17. | N | CH | H | Methoxy | n-Propyl |
| A-18. | N | CH | Ethoxy | Methoxy | n-Propyl |
| A-19. | N | CH | Methoxy | Methoxy | Isopropyl |
| A-20. | N | CH | Methoxy | H | Isopropyl |
| A-21. | N | CH | Ethoxy | H | Isopropyl |
| A-22. | N | CH | H | H | Isopropyl |
| A-23. | N | CH | H | Methoxy | Isopropyl |
| A-24. | N | CH | Ethoxy | Methoxy | Isopropyl |
| A-25. | N | CH | Methoxy | Methoxy | H |
| A-26. | N | CH | Methoxy | H | H |
| A-27. | N | CH | Ethoxy | H | H |
| A-28. | N | CH | H | H | H |
| A-29. | N | CH | H | Methoxy | H |
| A-30. | N | CH | Ethoxy | Methoxy | H |
| A-31. | CH | N | Methoxy | Methoxy | Methyl |
| A-32. | CH | N | Methoxy | H | Methyl |
| A-33. | CH | N | Ethoxy | H | Methyl |
| A-34. | CH | N | H | H | Methyl |
| A-35. | CH | N | H | Methoxy | Methyl |
| A-36. | CH | N | Ethoxy | Methoxy | Methyl |
| A-37. | CH | N | Methoxy | Methoxy | Ethyl |
| A-38. | CH | N | Methoxy | H | Ethyl |
| A-39. | CH | N | Ethoxy | H | Ethyl |
| A-40. | CH | N | H | H | Ethyl |
| A-41. | CH | N | H | Methoxy | Ethyl |
| A-42. | CH | N | Ethoxy | Methoxy | Ethyl |
| A-43. | CH | N | Methoxy | Methoxy | n-Propyl |
| A-44. | CH | N | Methoxy | H | n-Propyl |
| A-45. | CH | N | Ethoxy | H | n-Propyl |
| A-46. | CH | N | H | H | n-Propyl |
| A-47. | CH | N | H | Methoxy | n-Propyl |
| A-48. | CH | N | Ethoxy | Methoxy | n-Propyl |
| A-49. | CH | N | Methoxy | Methoxy | Isopropyl |
| A-50. | CH | N | Methoxy | H | Isopropyl |
| A-51. | CH | N | Ethoxy | H | Isopropyl |
| A-52. | CH | N | H | H | Isopropyl |
| A-53. | CH | N | H | Methoxy | Isopropyl |
| A-54. | CH | N | Ethoxy | Methoxy | Isopropyl |
| A-55. | CH | N | Methoxy | Methoxy | H |
| A-56. | CH | N | Methoxy | H | H |
| A-57. | CH | N | Ethoxy | H | H |
| A-58. | CH | N | H | H | H |
| A-59. | CH | N | H | Methoxy | H |
| A-60. | CH | N | Ethoxy | Methoxy | H |
| A-61. | CH | CH | Methoxy | Methoxy | Methyl |
| A-62. | CH | CH | Methoxy | H | Methyl |
| A-63. | CH | CH | Ethoxy | H | Methyl |
| A-64. | CH | CH | H | H | Methyl |
| A-65. | CH | CH | H | Methoxy | Methyl |
| A-66. | CH | CH | Ethoxy | Methoxy | Methyl |
| A-67. | CH | CH | Methoxy | Methoxy | Ethyl |
| A-68. | CH | CH | Methoxy | H | Ethyl |
| A-69. | CH | CH | Ethoxy | H | Ethyl |
| A-70. | CH | CH | H | H | Ethyl |
| A-71. | CH | CH | H | Methoxy | Ethyl |
| A-72. | CH | CH | Ethoxy | Methoxy | Ethyl |
| A-73. | CH | CH | Methoxy | Methoxy | n-Propyl |
| A-74. | CH | CH | Methoxy | H | n-Propyl |
| A-75. | CH | CH | Ethoxy | H | n-Propyl |
| A-76. | CH | CH | H | H | n-Propyl |
| A-77. | CH | CH | H | Methoxy | n-Propyl |
| A-78. | CH | CH | Ethoxy | Methoxy | n-Propyl |
| A-79. | CH | CH | Methoxy | Methoxy | Isopropyl |
| A-80. | CH | CH | Methoxy | H | Isopropyl |
| A-81. | CH | CH | Ethoxy | H | Isopropyl |
| A-82. | CH | CH | H | H | Isopropyl |
| A-83. | CH | CH | H | Methoxy | Isopropyl |
| A-84. | CH | CH | Ethoxy | Methoxy | Isopropyl |
| A-85. | CH | CH | Methoxy | Methoxy | H |
| A-86. | CH | CH | Methoxy | H | H |
| A-87. | CH | CH | Ethoxy | H | H |
| A-88. | CH | CH | H | H | H |
| A-89. | CH | CH | H | Methoxy | H |
| A-90. | CH | CH | Ethoxy | Methoxy | H |

The compounds preferred among the compounds I.1 to I.60 mentioned above are those of the formulae I.1, I.2, I.5, I.6, I.7, I,10, I.11, I.12, I.15, I.16, I.17, I.20, I.20, I.21, I.22, I.25, I.26, I.27, I.30, I.31, I.32, I.35, I.26, I.37, I.40, I.41, I.42, I.45, I.46, I.47, I.50, I.51, I.52, I.55, I.56, I.57 and I.60, in which the radicals X², X³, R¹, R² and R³ assume in each case the meanings mentioned in each line in table 1. Compounds among these which are in turn preferred are those of the formulae I.1, I.2, I.6, I.7and I.10, in which the radicals X², X³, R¹, R² and R³ assume in each case the meanings mentioned in each line in table 1. Compounds more preferred among these are those of the formulae I.1, I.2 and I.5, in which the radicals X², X³, R¹, R² and R³ assume in each case the meanings mentioned in each line in table 1. Compounds particularly preferred among these are those in which the radicals X², X³, R¹, R² and R³ assume in each case the meanings mentioned in table 1 in lines A-1, A-7, A-31 and A-37.The compounds preferred among the compounds I.61 to I.78 mentioned above are those of the formulae I.61, I.62, I.67 and I.68, in which the radicals X², X³, R¹, R² and R³ assume in each case the meanings mentioned in each line in table 1. Compounds among these which are in turn preferred are those of the formulae I.61 and I.62, in which the radicals X², X³, R¹, R² and R³ assume in each case the meanings mentioned in each line in table 1. Among these, compounds particularly preferred are those, in which the radicals X², X³, R¹, R² and R³ assume in each case the meanings mentioned in table 1 in lines A-1, A-7, A-31 and A-37. Moreover, the compound of formula I.79 is particularly preferred, especially the compound I.79 in which the radicals X², X³, R¹, R² and R³ assume in each case the meanings mentioned in table 1 in lines A-1, A-7, A-31 and A-37.

The patient to be treated prophylactically or therapeutically according to the method of the invention is preferably a mammal, for example a human or a nonhuman mammal or a nonhuman transgenic mammal. Specifically it is a human.

The compounds of the general formula I, their pharmaceutically acceptable salts and prodrugs as detailed above can be prepared by a skilled worker with knowledge of the technical teaching of the invention in implementing and/or in analogous implementation of process steps known per se.

The compounds I or their prodrugs and/or their pharmaceutically acceptable salts are distinguished by having a selectivity for the vasopressin V1b receptor subtype vis-à-vis at least one of the closely related vasopressin/oxytocin receptor subtypes (for example vasopressin V1a, vasopressin V2 and/or oxytocin).

Alternatively, or preferably in addition, the compounds I or their prodrugs and/or their pharmaceutically acceptable salts are distinguished by having an improved metabolic stability.

The metabolic stability of a compound can be measured for example by incubating a solution of this compound with liver microsomes from particular species (for example rat, dog or human) and determining the half-life of the compound under these conditions (RS Obach, Curr Opin Drug Discov Devel. 2001, 4, 36-44). It is possible in this connection to conclude from an observed longer half-life that the metabolic stability of the compound is improved. The stability in the presence of human liver microsomes is of particular interest because it makes it possible to predict the metabolic degradation of the compound in the human liver. Compounds with increased metabolic stability (measured in the liver microsome test) are therefore probably also degraded more slowly in the liver. The slower metabolic degradation in the liver may lead to higher and/or longer-lasting concentrations (active levels) of the compound in the body, so that the elimination half-life of the compounds of the invention is increased. Increased and/or longer-lasting active levels may lead to a better activity of the compound in the treatment or prophylaxis of various vasopressin-dependent diseases. In addition, an improved metabolic stability may lead to an increased bioavailability after oral administration, because the compound is subject, after absorption in the intestine, to less metabolic degradation in the liver (so-called first pass effect). An increased oral bioavailability may, owing to an increased concentration (active level) of the compound, lead to a better activity of the compound after oral administration.

Alternatively, or preferably in addition, the compounds I or their prodrugs and/or their pharmaceutically acceptable salts are distinguished by having an improved pharmacological activity, compared with other analgesic compounds known from the prior art, in patients or relevant animal models which enable prognostic statements for use in the treatment.

The compounds used according to the invention are effective after administration by various routes. Possible examples are intravenous, intramuscular, subcutaneous, topical, intratracheal, intranasal, transdermal, vaginal, rectal, sublingual, buccal or oral administration, and administration is frequently intravenous, intramuscular or, in particular, oral.

The present invention also relates to pharmaceutical compositions which comprise an effective dose of a compound I of the invention, of a pharmaceutically acceptable salt or of a prodrug thereof and suitable pharmaceutical carriers (drug carriers) and its use in the method of the invention.

These drug carriers are chosen according to the pharmaceutical form and the desired mode of administration and are known in principle to the skilled worker.

The compounds of the formula I or optionally suitable salts of these compounds can be used to produce pharmaceutical compositions for oral, sublingual, buccal, subcutaneous, intramuscular, intravenous, topical, intratracheal, intranasal, transdermal, vaginal or rectal administration, and be administered to animals or humans in uniform administration forms, mixed with conventional pharmaceutical carriers, for the prophylaxis or treatment of the above disorders or diseases.

The suitable administration forms (dose units) include forms for oral administration such as tablets, gelatin capsules, powders, granules and solutions or suspensions for oral intake, forms for sublingual, buccal, intratracheal or intranasal administration, aerosols, implants, forms of subcutaneous, intramuscular or intravenous administration and forms of rectal administration.

The compounds I can be used in creams, ointments or lotions for topical administration.

In order to achieve the desired prophylactic or therapeutic effect, the dose of the active ingredient can vary between 0.01 and 50 mg per kg of body weight and per day.

Each unit dose may comprise from 0.05 to 5000 mg, preferably 1 to 1000 mg, of the active ingredient in combination with a pharmaceutical carrier. This unit dose can be administered once to 5 times a day, so that a daily dose of from 0.5 to 25 000 mg, preferably 1 to 5000 mg, is administered.

If a solid composition is prepared in the form of tablets, the active ingredient is mixed with a solid pharmaceutical carrier such as gelatin, starch, lactose, magnesium stearate, talc, silicon dioxide or the like.

The tablets can be coated with sucrose, a cellulose derivative or another suitable substance or be treated otherwise in order to display a sustained or delayed activity and to release a predetermined amount of the active ingredient continuously.

A preparation in the form of gelatin capsules is obtained by mixing the active ingredient with an extender and including the resulting mixture in soft or hard gelatin capsules.

A preparation in the form of a syrup or elixir or for administration in the form of drops may contain active ingredients together with a sweetener, which is preferably calorie-free, methylparaben or propylparaben as antiseptics, a flavoring and a suitable coloring substance.

Water-dispersible powders or granules may comprise the active ingredients mixed with dispersants, wetting agents or suspending agents, such as polyvinylpyrrolidones, and sweeteners or masking flavors.

Rectal or vaginal administration is achieved by using suppositories which are prepared with binders which melt at rectal temperature, for example cocoa butter or polyethylene glycols. Parenteral administration is effected by using aqueous suspensions, isotonic saline solutions or sterile and injectable solutions which comprise pharmacologically acceptable dispersants and/or wetting agents, for example propylene glycol or polyethylene glycol.

The active ingredient may also be formulated as microcapsules or centrosomes, if suitable with one or more carriers or additives.

The present invention moreover relates to compounds of formula I as defined above, wherein at least one of the atoms has been replaced by its stable, non-radioactive isotope (e.g., hydrogen by deuterium, ¹³C by ¹³C, ¹⁴N by ¹⁵N, ¹⁶O by ¹⁸O) and preferably wherein at least one hydrogen atom has been replaced by a deuterium atom.

Of course, the compounds according to the invention contain more of the respective isotope than this naturally occurs and thus is anyway present in the compounds I.

Stable isotopes (e.g., deuterium, ¹³C, ¹⁵N, ¹⁸O) are nonradioactive isotopes which contain one additional neutron than the normally abundant isotope of the respective atom. Deuterated compounds have been used in pharmaceutical research to investigate the in vivo metabolic fate of the compounds by evaluation of the mechanism of action and metabolic pathway of the non deuterated parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

Incorporation of a heavy atom particularly substitution of deuterium for hydrogen, can give rise to an isotope effect that could alter the pharmacokinetics of the drug. This effect is usually insignificant if the label is placed at a metabolically inert position of the molecule.

Stable isotope labeling of a drug can alter its physico-chemical properties such as pKa and lipid solubility. These changes may influence the fate of the drug at different steps along its passage through the body. Absorption, distribution, metabolism or excretion can be changed. Absorption and distribution are processes that depend primarily on the molecular size and the lipophilicity of the substance. These effects and alterations can affect the pharmacodynamic response of the drug molecule if the isotopic substitution affects a region involved in a ligand-receptor interaction.

Drug metabolism can give rise to large isotopic effect if the breaking of a chemical bond to a deuterium atom is the rate limiting step in the process. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. In any reaction in which the breaking of this bond is the rate limiting step, the reaction will proceed slower for the molecule with the heavy isotope due to “kinetic isotope effect”. A reaction involving breaking a C—D bond can be up to 700 percent slower than a similar reaction involving breaking a C—H bond. If the C—D bond is not involved in any of the steps leading to the metabolite, there may not be any effect to alter the behavior of the drug. If a deuterium is placed at a site involved in the metabolism of a drug, an isotope effect will be observed only if breaking of the C—D bond is the rate limiting step. There is evidence to suggest that whenever cleavage of an aliphatic C—H bond occurs, usually by oxidation catalyzed by a mixed-function oxidase, replacement of the hydrogen by deuterium will lead to observable isotope effect. It is also important to understand that the incorporation of deuterium at the site of metabolism slows its rate to the point where another metabolite produced by attack at a carbon atom not substituted by deuterium becomes the major pathway a process called “metabolic switching”.

Deuterium tracers, such as deuterium-labeled drugs and doses, in some cases repeatedly, of thousands of milligrams of deuterated water, are also used in healthy humans of all ages, including neonates and pregnant women, without reported incident (e.g. Pons G and Rey E, Pediatrics 1999 104: 633; Coward W A et al., Lancet 1979 7: 13; Schwarcz H P, Control. Clin. Trials 1984 5(4 Suppl): 573; Rodewald L E et al., J. Pediatr. 1989 114: 885; Butte N F et al. Br. J. Nutr. 1991 65: 3; MacLennan A H et al. Am. J. Obstet Gynecol. 1981 139: 948). Thus, it is clear that any deuterium released, for instance, during the metabolism of compounds of this invention poses no health risk.

The weight percentage of hydrogen in a mammal (approximately 9%) and natural abundance of deuterium (approximately 0.015%) indicates that a 70 kg human normally contains nearly a gram of deuterium. Furthermore, replacement of up to about 15% of normal hydrogen with deuterium has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci 1960 84: 736; Czakja D M et al., Am. J. Physiol. 1961 201: 357). Higher deuterium concentrations, usually in excess of 20%, can be toxic in animals. However, acute replacement of as high as 15%-23% of the hydrogen in humans' fluids with deuterium was found not to cause toxicity (Blagojevic N et al. in “Dosimetry & Treatment Planning for Neutron Capture Therapy”, Zamenhof R, Solares G and Harling O Eds. 1994. Advanced Medical Publishing, Madison Wis. pp.125-134; Diabetes Metab. 23: 251 (1997)).

Increasing the amount of deuterium present in a compound above its natural abundance is called enrichment or deuterium-enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %.

The hydrogens present on a particular organic compound have different capacities for exchange with deuterium. Certain hydrogen atoms are easily exchangeable under physiological conditions and, if replaced by deuterium atoms, it is expected that they will readily exchange for protons after administration to a patient. Certain hydrogen atoms may be exchanged for deuterium atoms by the action of a deuteric acid such as D₂SO₄/D₂O. Alternatively, deuterium atoms may be incorporated in various combinations during the synthesis of compounds of the invention. Certain hydrogen atoms are not easily exchangeable for deuterium atoms. However, deuterium atoms at the remaining positions may be incorporated by the use of deuterated starting materials or intermediates during the construction of compounds of the invention.

Deuterated and deuterium-enriched compounds of the invention can be prepared by using known methods described in the literature. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Lizondo, J et al., Drugs Fut, 21(11), 1116 (1996); Brickner, S J et al., J Med Chem, 39(3), 673 (1996); Mallesham, Bet al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; 20090082471, the methods are hereby incorporated by reference.

The invention is explained in more detail below by means of examples, but the examples are not to be understood to be restrictive.

### Examples

Following Compound 1 was Tested:

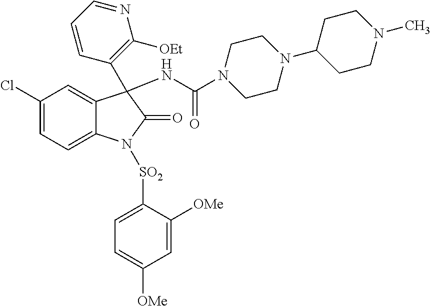

### Example 1

SNL surgery: For spinal nerve ligation surgery, rats were anesthetized with isoflurane gas. When the rat did not respond to tail pinch the surgery begins. The hair at the surgical site was clipped and disinfected with alcohol and betadine. Body temperature was maintained during surgery by using a heating pad. A skin incision (approximately 3 cm) was made on the dorsal midline, using the level of the iliac crests as the midpoint of the incision. At the mid-sacral region, on the left side of the vertebral column (in the sagittal plane), a #15 scalpel blade was used to cut the muscles close to the vertebral body until the blade hits the sacrum bone. A retractor system was used to expose the area from the sacro-iliac rim to about 2 cm of the lateral vertebral column. Using rongeurs, the facet joint was removed followed by the L6 transverse process. A glass hook was used to isolate and tightly ligate L5 and L6 with 6-0 silk suture. The muscle was then sutured using 4-0 silk suture. The skin was closed using wound clips.

Tactile allodynia test: The rats were placed on testing stands with screens under their feet. von Frey filaments eliciting various forces (up to 15 g) were applied to the paws, starting with a filament of 4.31 g and proceeding with alternating lighter and heavier filaments. The measured variable is the 50% withdrawal threshold (PWT) and is calculated from the up-down formula in Chaplan et al. 1994.

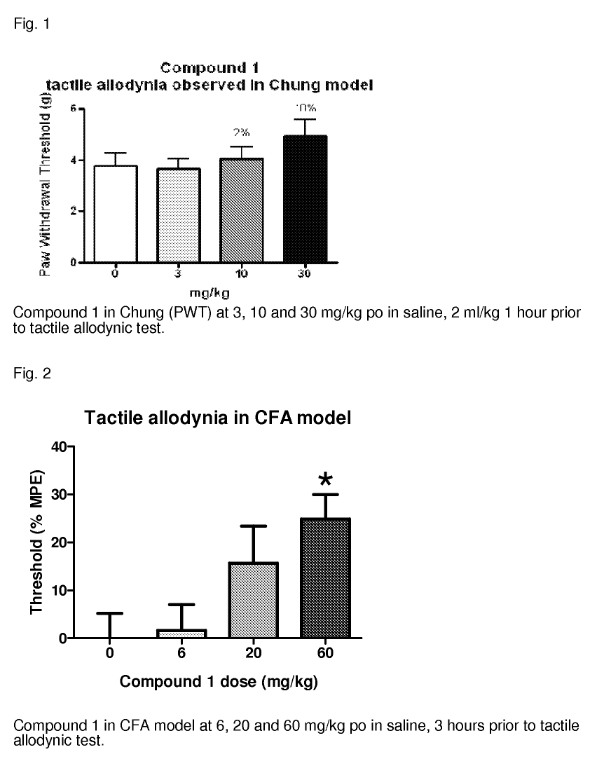

The results are shown in FIG. 1.

FIG. 1 shows the paw withdrawl threshold obtained with compound 1 in Chung (PWT) at 3, 10 and 30 mg/kg po in saline, 2 ml/kg, administered 1 hour prior to the tactile allodynic test.

As the results show, compound 1 dose-dependently increased the withdrawal threshold in the Chung model. The results were obtained only 1 h after the administration of compound 1.

### Example 2

Complete Freund's Adjuvant (CFA) Protocol

Prior to behavioral testing (2 days) for inflammation and hyperalgesia, each rat was briefly restrained and given an intraplantar injection of Complete Freund's Adjuvant (150 ul of 0.5-1.0 mg/ml solutions) with the CFA solution either undiluted or diluted 1:1 in phosphate buffered saline (PBS). This model induces a unilateral inflammation and edema localized to the injected hindpaw. The inflammation has a slow onset and peaks 2-3 days post intraplantar CFA administration and lasts for 10 days or longer. Plasticity in the nociceptive receptive fields has been shown by electrophysiological recording from dorsal horn neurons before injection and then during the development of the inflammation and hyperalgesia, showing a progressive enlargement of pain-responsive neuronal receptive fields.

Tactile allodynia was measured using calibrated von Frey filaments (Stoelting, Wood Dale, Ill.) as previously described (Chaplan et al., 1994). Rats were placed into inverted individual plastic containers (20×12.5×20 cm) on top of a suspended wire mesh grid, and acclimated to the test chambers for 20 min. The von Frey filaments with different bending forces (starting with the lowest first and then progressively increasing) were presented perpendicularly to the plantar surface of the selected hind paw, and then held in this position for approximately 8 sec with enough force to cause a slight bend in the filament. Positive responses included an abrupt withdrawal of the hind paw from the stimulus, or flinching behavior immediately following removal of the stimulus. The maximum force applied was 15 g, which is a force that normally does not evoke a response in a naive rat. Typically, only rats that exhibit an altered state (allodynia/hyperalgesia) have responded to stimulation from fibers that exert a force of less than 15 g. Normally, the force of the von Frey hairs is innocuous, only in an altered state (allodynic, hyperalgesic) do the animals respond to this stimulation. Rats with thresholds scores less than or equal to 5 are considered allodynic and utilized for further testing.

Compound 1 was prepared in saline and administered per os to fasted rats 48 hours after the injection of CFA.

The results are shown in FIG. 2.

FIG. 2 shows tactile thresholds (expressed as % from maximal possible effect) measured three hours after the administration of compound 1 at 6, 20 and 60 mg/kg po in saline (N=6 per data point). For analysis, data were subjected to Krustal-Wallis' test followed by Dunn's test for pairwise between-group comparisons (* P<0.05).

1. A method for the treatment or prophylaxis of pain, comprising administering to a subject in need thereof a pharmaceutically acceptable salt or a prodrug of a compound of the formula I 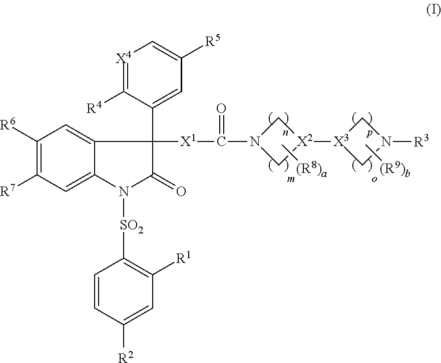
in which
R¹ and R² are independently of one another hydrogen, C₁-C₃-alkyl, C₁-C₃-fluoroalkyl, C₁-C₃-alkoxy, C₁-C₃-fluoroalkoxy, halogen or CN;
R³ is hydrogen or C₁-C₄-alkyl;
R⁴ is methoxy, ethoxy, fluorinated ethoxy or isopropoxy;
R⁵ is hydrogen or methyl;
R⁶ is Br, Cl, F or CN;
R⁷ is hydrogen, Cl, F or CN;
R⁸ and R⁹ are independently of one another C₁-C₃-alkyl or C₁-C₃-fluoroalkyl;
X¹ is O, NH or CH₂;
X² and X³ are N or CH, with the proviso that X² and X³ are not simultaneously N;
X⁴ is N or CH;
a and b are independently of one another 0, 1 or 2; and
m, n, o and p are independently of one another 1, 2 or 3;
or of a pharmaceutically acceptable salt thereof or of a prodrug thereof;
for preparing a medicament for the treatment or prophylaxis of pain.

2. The method as claimed in claim 1, wherein the pain is chronic pain.

3. The method as claimed in claim 2, where the chronic pain is neuropathic pain.

4. The method as claimed in claim 1, where R¹ is hydrogen, methoxy, ethoxy, fluoromethoxy, difluoromethoxy or trifluoromethoxy.

5. (canceled)

6. The method as claimed in claim 1, where R¹ is hydrogen or methoxy.

7. The method as claimed in claim 1, where R² is hydrogen or methoxy.

8-9. (canceled)

10. The method as claimed in claim 1, where R³ is hydrogen, methyl or ethyl.

11. (canceled)

12. The method as claimed in claim 1 where R⁴ is ethoxy and R⁵ is H; R⁴ is 2,2-difluoroethoxy or 2,2,2-trifluoroethoxy and R⁵ is H, where R⁴ is ethoxy and R⁵ is methyl, where R⁴ is isopropoxy and R⁵ is H, or where R⁴ is methoxy and R⁵ is H.

13-18. (canceled)

19. The method as claimed in claim 1, where R⁷ is F and R⁶ is F, Cl, Br or CN, where R⁶ is F or Cl and R⁷ is hydrogen, and where R⁶ is CN and R⁷ is hydrogen.

20-22. (canceled)

23. The method as claimed in claim 1, where R⁸ and R⁹ are methyl or ethyl.

24. The method as claimed in claim 1, where X² is N and X³ is CH.

25. The method as claimed in claim 1, where X² is CH and X³ is N.

26. The method as claimed in claim 1, where X² is CH and X³ is CH.

27-29. (canceled)

30. The method as claimed in claim 1, where X⁴ is N.

31. The method as claimed in claim 1, where a and b are 0.

32. The method as claimed in claim 1, where m, n, o and p are 2.

33. The method as claimed in claim 1, of a compound of the formula I.A 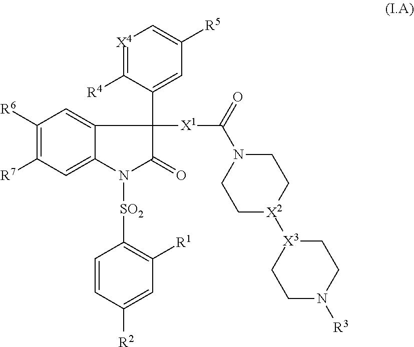
in which R¹, R², R³, R⁴, R⁵, R⁶, R⁷, X¹, X², X³ and X⁴ have the meanings indicated claim 1.

34. The method as claimed in claim 33, where
R¹ is methoxy;
R² is methoxy;
R³ is methyl or ethyl;
R⁴ is ethoxy;
R⁵ is H;
R⁶ is Cl or CN;
R⁷ is F;
X¹ is NH;
X² is N;
X³ is CH; and
X⁴ is N;
or
R¹ is methoxy;
R² is methoxy;
R³ is methyl or ethyl;
R⁴ is ethoxy;
R⁵ is H;
R⁶ is Cl or CN;
R⁷ is F;
X¹ is NH;
X² is CH;
X³ is N; and
X⁴ is N;
or
R¹ is methoxy;
R² is methoxy;
R³ is methyl or ethyl;
R⁴ is ethoxy;
R⁵ is H;
R⁶ is Cl or CN;
R⁷ is F;
X¹ is CH₂;
X² is N;
X³ is CH; and
X⁴ is N;
or
R¹ is methoxy;
R² is methoxy;
R³ is methyl or ethyl;
R⁴ is ethoxy;
R⁵ is H;
R⁶ is Cl or CN;
R⁷ is F;
X¹ is CH₂;
X² is CH;
X³ is N; and
X⁴ is N;
or
R¹ is methoxy or H;
R² is methoxy;
R³ is methyl or ethyl;
R⁴ is ethoxy;
R⁵ is H;
R⁶ is Cl or CN;
R⁷ is F;
X¹ is O;
X² is N;
X³ is CH; and
X⁴ is N;
or
R¹ is methoxy or H;
R² is methoxy;
R³ is methyl or ethyl;
R⁴ is ethoxy;
R⁵ is H;
R⁶ is Cl or CN;
R⁷ is F;
X¹ is O;
X² is CH;
X³ is N; and
X⁴ is N;
or
R¹ is methoxy;
R² is methoxy;
R³ is methyl or ethyl;
R⁴ is ethoxy;
R⁵ is H;
R⁶ is Cl or CN;
R⁷ is F;
X¹ is NH;
X² is N;
X³ is CH; and
X⁴ is CH;
or
R¹ is methoxy;
R² is methoxy;
R³ is methyl or ethyl;
R⁴ is ethoxy;
R⁵ is H;
R⁶ is F;
R⁷ is F;
X¹ is NH;
X² is N;
X³ is CH; and
X⁴ is N;
or
R¹ is methoxy;
R² is methoxy;
R³ is methyl or ethyl;
R⁴ is ethoxy;
R⁵ is H;
R⁶ is F;
R⁷ is F;
X¹ is NH;
X² is CH;
X³ is N; and
X⁴ is N;
or
R¹ is methoxy;
R² is methoxy;
R³ is methyl;
R⁴ is ethoxy;
R⁵ is H;
R⁶ is Cl;
R⁷ is H;
X¹ is NH;
X² is N;
X³ is CH; and
X⁴ is N;
or
R¹ is methoxy;
R² is methoxy;
R³ is methyl;
R⁴ is ethoxy;
R⁵ is methyl;
R⁶ is Cl;
R⁷ is H;
X¹ is NH;
X² is N;
X³ is CH; and
X⁴ is N;
or
R¹ is methoxy;
R² is methoxy;
R³ is ethyl;
R⁴ is ethoxy;
R⁵ is H;
R⁶ is Cl;
R⁷ is H;
X¹ is NH;
X² is N;
X³ is CH; and
X⁴ is N;
or
R¹ is methoxy;
R² is methoxy;
R³ is methyl;
R⁴ is ethoxy;
R⁵ is H;
R⁶ is Cl;
R⁷ is H;
X¹ is NH;
X² is CH;
X³ is N; and
X⁴ is N;
or
R¹ is methoxy;
R² is methoxy;
R³ is methyl;
R⁴ is methoxy;
R⁵ is H;
R⁶ is Cl;
R⁷ is H;
X¹ is NH;
X² is N;
X³ is CH; and
X⁴ is N;
or
R¹ is methoxy;
R² is methoxy;
R³ is methyl;
R⁴ is methoxy;
R⁵ is H;
R⁶ is Cl;
R⁷ is H;
X¹ is NH;
X² is CH;
X³ is N; and
X⁴ is N;
or
R¹ is methoxy;
R² is methoxy;
R³ is ethyl;
R⁴ is methoxy;
R⁵ is H;
R⁶ is Cl;
R⁷ is H;
X¹ is NH;
X² is N;
X³ is CH; and
X⁴ is N;
or
R¹ is methoxy;
R² is methoxy;
R³ is ethyl;
R⁴ is methoxy;
R⁵ is H;
R⁶ is Cl;
R⁷ is H;
X¹ is NH;
X² is CH;
X³ is N; and
X⁴ is N;
or
R¹ is methoxy;
R² is methoxy;
R³ is methyl;
R⁴ is ethoxy;
R⁵ is H;
R⁶ is CN;
R⁷ is H;
X¹ is CH₂;
X² is CH;
X³ is N; and
X⁴ is N;
or
R¹ is methoxy;
R² is methoxy;
R³ is methyl;
R⁴ is ethoxy;
R⁵ is H;
R⁶ is CN;
R⁷ is H;
X¹ is CH₂;
X² is N;
X³ is CH; and
X⁴ is N;
or
R¹ is methoxy;
R² is methoxy;
R³ is methyl;
R⁴ is ethoxy;
R⁵ is H;
R⁶ is CN;
R⁷ is H;
X¹ is CH₂;
X² is CH;
X³ is CH; and
X⁴ is N;
or
R¹ is methoxy;
R² is methoxy;
R³ is ethyl;
R⁴ is ethoxy;
R⁵ is H;
R⁶ is CN;
R⁷ is H;
X¹ is CH₂;
X² is CH;
X³ is N; and
X⁴ is N;
or
R¹ is methoxy;
R² is methoxy;
R³ is ethyl;
R⁴ is ethoxy;
R⁵ is H;
R⁶ is CN;
R⁷ is H;
X¹ is CH₂;
X² is N;
X³ is CH; and
X⁴ is N;
or
R¹ is methoxy;
R² is methoxy;
R³ is methyl;
R⁴ is methoxy;
R⁵ is H;
R⁶ is CN;
R⁷ is H;
X¹ is NH;
X² is N;
X³ is CH; and
X⁴ is N;
or
R¹ is methoxy;
R² is methoxy;
R³ is methyl;
R⁴ is methoxy;
R⁵ is H;
R⁶ is CN;
R⁷ is H;
X¹ is NH;
X² is CH;
X³ is N; and
X⁴ is N;
or
R¹ is methoxy;
R² is methoxy;
R³ is ethyl;
R⁴ is methoxy;
R⁵ is H;
R⁶ is CN;
R⁷ is H;
X¹ is NH;
X² is N;
X³ is CH; and
X⁴ is N;
or
R¹ is methoxy;
R² is methoxy;
R³ is ethyl;
R⁴ is methoxy;
R⁵ is H;
R⁶ is CN;
R⁷ is H;
X¹ is NH;
X² is CH;
X³ is N; and
X⁴ is N.

35-60. (canceled)

61. A compound of formula I as defined in claim 1, wherein at least one of the atoms has been replaced by its stable, non-radioactive isotope.

62. A compound of formula I as defined in claim 61, wherein at least one hydrogen atom has been replaced by a deuterium atom.
